(19) United States
(12) Patent Application Publication
(10) Pub. No.: US 2023/0245773 A1
(43) Pub. Date: Aug. 3, 2023

(54) SYSTEM AND METHOD FOR DETECTING OR PREDICTING RETURN IN MAJOR DEPRESSIVE DISORDER

(71) Applicant: JANSSEN PHARMACEUTICA NV, BEERSE (BE)

(72) Inventors: SRINIVASAN VAIRAVAN, CRANBURY, NJ (US); VAIBHAV NARAYAN, PRINCETON, NJ (US); RANDALL MORRISON, GLENSIDE, PA (US)

(73) Assignee: JANSSEN PHARMACEUTICA NV, BEERSE (BE)

(21) Appl. No.: 18/002,527

(22) PCT Filed: Jul. 6, 2021
(86) PCT No.: PCT/EP2021/068689

§ 371 Date: Dec. 20, 2022

Related U.S. Application Data
(60) Provisional application No. 63/202,871, filed on Jun. 28, 2021.
(60) Provisional application No. 63/049,053, filed on Jul. 7, 2020.

(51) Int. Cl. G16H 50/20; G16H 20/70; G16H 10/20; G16H 20/10

(52) U.S. Cl. CPC G16H 50/20; G16H 20/70; G16H 10/20; G16H 20/10

(57) ABSTRACT

A system and computer-implemented method for detecting return of depression of a patient is provided. The system comprises a wearable device configured to detect movement of the patient and configured to generate actigraphy data corresponding to the movement of the patient and a computing device for retrieving actigraphy data from the device. The system and method obtain training data, including training actigraphy data, over a training period and train an anomaly detector using the training data. The system and method subsequently obtain test data from the patient, extract a plurality of features from the test data, and analyze the extracted data using the trained anomaly detector. A self-report test is used to determine whether an anomaly identified by the anomaly detector indicates that the patient is likely to experience return of depression.

### PRIORITY CLAIM

This application claims priority to U.S. Provisional Application Ser. No. 63/049,053 filed Jul. 7, 2020, and to U.S. Provisional Application Ser. No. 63/202,871 filed Jun. 28, 2021, the entire contents of which are hereby incorporated by reference herein.

### BACKGROUND OF THE INVENTION

Major Depressive Disorder (MDD) is one of the leading causes of disability worldwide (measured as years lived with disability), with a lifetime prevalence of approximately 15% in the general adult population and is associated with significant morbidity and mortality. This condition affects more than 300 million people worldwide. Patients who suffer from MDD can experience a wide range of physical, emotional and cognitive symptoms including depressed mood, loss of interest or pleasure in all/almost all activities, fatigue and sleep disruption, and difficulties with thinking, concentrating and making decisions. These symptoms can severely impact patients' daily lives, including how they feel, think and handle daily activities, and can affect their health, relationships, employment, education, and overall quality of life. In severe cases of MDD, patients may have thoughts of death or suicide. Notably, those suffering from MDD have a 20-fold higher risk of suicide than the general population. Furthermore, MDD is believed to contribute to an increased risk of developing or worsening of other health disorders. For example, MDD can increase the risk of developing conditions such as stroke and type 2 diabetes.

There are various treatment options that help patients relieve symptoms of MDD and improve their quality of life. However, even with treatment, MDD is a chronic disorder with recurrent episodes, such that patients may experience residual symptoms, or experience relapse or recurrence of depression. In clinical practice, clinicians take a reactive approach by observing patients only during clinical visits and making changes in patients' treatment regimens as needed in response to observations made during such clinical visits. MDD is a dynamic illness, with episodes of relapse interspersed with periods of remission. Transitions in disease states may arise on timescales faster than the time between physician visits. Using this reactive approach, the clinicians are often unaware of early changes in patients' symptomatology. Relapse or recurrence is frequently only detected after a patient's depressive symptoms have worsened enough to warrant a clinical visit for assessment.

Delays in obtaining further treatment after a relapse or a recurrence may put patients at a higher risk for self-harm or suicide. The proportion of MDD patients who achieve remission also decreases significantly after each treatment failure. Furthermore, lengthy and/or ineffective treatments can prolong patient suffering, reduce expectations, and reinforce negative emotions such as hopelessness. Therefore, early identification and recognition of a relapse or a recurrence into depression, could enable clinicians to intercept disease worsening earlier, be potentially lifesaving, and improve patients' opportunity to attain meaningful response to treatment and potentially reach remission.

### BRIEF SUMMARY OF THE INVENTION

One exemplary embodiment of the present invention is directed to a computer-implemented method for detecting or predicting return of depression in a patient. The method comprises (i) obtaining, from a wearable device worn by the patient, training data of the patient over a training period. The training data comprises training actigraphy data corresponding to movement of the patient over the training period. The training period is during a time period when the patient has not experienced onset of return of depression. The method also comprises (ii) training an anomaly detector using the training data. The anomaly detector is configured to identify deviations from the training data. The method further comprises (iii) obtaining, from the wearable device, test data of the patient during a test period after the training period. The test data comprising test actigraphy data corresponding to movement of the patient after the training period. The method further comprises (iv) extracting a plurality of features from the test data to generate test feature data, wherein the features correspond to metrics for at least one of activity, sleep, circadian rhythm, and multifractal dynamics. The method further comprises (v) analyzing the test feature data using the anomaly detector to compare the test feature data to the training data. The method further comprises (vi) administering a self-report test to the patient to obtain a plurality of inputs from the patient when the anomaly detector determines that the test feature data is likely an anomaly compared to the training actigraphy data. The method further comprises (vii) analyzing the plurality of inputs from the patient to determine whether the patient is likely to experience onset of return of depression.

A system for detecting or predicting return of depression in a patient is also provided. The system comprises a wearable device comprising at least one accelerometer configured to detect movement of the patient. The wearable device configured to generate actigraphy data corresponding to movement of the patient. The system also comprises a computing device operably connected to the wearable device to receive actigraphy data from the wearable device. The computing device comprises a user interface for displaying output and receiving input from the patient, and a processor and a non-transitory computer readable storage medium including a set of instructions executable by the processor. The set of instructions are operable to: obtain, from the wearable device, training actigraphy data corresponding to movement of the patient over a training period, wherein the training period is during a time period when the patient has not experienced onset of return of depression, train an anomaly detector using training data comprising the training actigraphy data, wherein the anomaly detector is configured to identify deviations from the training data, obtaining, from the wearable device, test actigraphy data corresponding to movement of the patient after the training period, extract a plurality of features from the test actigraphy data to generate test feature data, wherein the features correspond to metrics for at least one of activity, sleep, circadian rhythm, and multifractal dynamics analyze the test feature data using the anomaly detector to compare the test feature data to the training data, direct the user interface to display a plurality of self-report survey questions to the patient, receive, via the user interface, the plurality of inputs from the patient in response to the self-report survey questions, and analyze the plurality of inputs from the patient to determine whether the patient is likely to experience onset of return of depression.

In another aspect, a computer-implemented method for detecting or predicting return of depression in a patient is provided. The method comprises (i) obtaining, from a wearable device worn by the patient, training data of the patient over a training period. The training data comprises training actigraphy data corresponding to movement of the patient over the training period, and the training period is during a time period when the patient has not experienced onset of return of depression. The method also comprises (ii) training an anomaly detector using the training data, wherein the anomaly detector is configured to identify deviations from the training data. The method further comprises (iii) obtaining, from the wearable device, test data of the patient during a test period. At least a portion of the test period is after the training period. The test data comprise test actigraphy data corresponding to movement of the patient after the training period. The method further comprises (iv) extracting a plurality of features from the test data to generate test feature data, wherein the features correspond to metrics for at least one of monofractal patterns, multifractal dynamics and sample entropy. The method further comprises (v) analyzing the test feature data using the anomaly detector to compare the test feature data to the training data to detect an anomaly in the test feature data, and (vi) analyzing self-report test data to determine whether the patient is likely to experience onset of return of depression when an anomaly is detected in the test feature data, wherein the self-report test data is generated from a plurality of inputs from the patient in response to a self-report test.

In a further aspect, a system for detecting or predicting return of depression in a patient is provided. The system comprises a wearable device comprising at least one accelerometer configured to detect movement of the patient, the wearable device configured to generate actigraphy data corresponding to movement of the patient. The system also comprises a computing device operably connected to the wearable device to receive actigraphy data from the wearable device. The computing device comprises a user interface for displaying output and receiving input from the patient, and a processor and a non-transitory computer readable storage medium including a set of instructions executable by the processor. The set of instructions are operable to obtain, from the wearable device, training actigraphy data corresponding to movement of the patient over a training period, wherein the training period is during a time period when the patient has not experienced onset of return of depression, train an anomaly detector using training data comprising the training actigraphy data, wherein the anomaly detector is configured to identify deviations from the training data, obtaining, from the wearable device, test actigraphy data corresponding to movement of the patient during a test period, at least a portion of the test period being after the training period, extract a plurality of features from the test actigraphy data to generate test feature data, wherein the features correspond to metrics for at least one of activity, for at least one of monofractal patterns, multifractal dynamics and sample entropy, analyze the test feature data using the anomaly detector to compare the test feature data to the training data to detect an anomaly in the test feature data, and analyze self-report test data to determine whether the patient is likely to experience onset of return of depression when an anomaly is detected in the test feature data. The self-report test data is generated from a plurality of inputs received from the patient by the user interface in response to a self-report test comprising a plurality of self-report survey questions displayed on the user interface.

These and other aspects of the invention will become apparent to those skilled in the art after a reading of the following detailed description of the invention, including the figures and appended claims.

### BRIEF DESCRIPTION OF THE DRAWINGS

FIG. 1 shows an exemplary system for detecting and/or predicting relapse of depression of a patient, according to an exemplary embodiment of the present application.

FIG. 2 shows an exemplary method for detecting and/or predicting relapse of depression of a patient, according to an exemplary embodiment of the present application.

FIG. 3 shows an exemplary method for training at least one anomaly detector for identifying deviations from training data to determine whether onset of relapse of depression is likely to occur based on passive patient data, according to an exemplary embodiment of the present application.

FIG. 4 shows an exemplary method for administering at least one self-report test to the patient and analyzing results of the self-report test(s) to further determine whether the patient is likely to experience onset of relapse of depression, according to an exemplary embodiment of the present application.

FIG. 5 shows an alternative exemplary method for administering at least one self-report test to the patient and analyzing results of the self-report test(s) to further determine whether the patient is likely to experience onset of relapse of depression, according to an exemplary embodiment of the present application.

FIG. 6 shows an exemplary a schematic of a LSTM anomaly detector having an encoder and a decoder.

FIG. 7 shows an exemplary timeline, as would be experienced by a patient, of the exemplary method of FIG. 2 for detecting and/or predicting relapse of depression of a patient.

FIG. 8 shows another exemplary method for detecting and/or predicting relapse of depression of a patient, according to an exemplary embodiment of the present application.

FIG. 9 shows an exemplary method for identifying anomalies using dynamic thresholds.

FIGS. 10a-f show an example of a time series of anomaly scores analyzed according to the steps of the exemplary method of FIG. 8.

FIG. 11 shows an example of an implementation of the exemplary method of FIG. 8 across a sample time series of data.

FIG. 12 shows an exemplary timeline, as would be experienced by a patient, of the exemplary method of FIG. 8 for detecting and/or predicting relapse of depression of a patient.

FIG. 13 shows an exemplary timeline for collecting training actigraphy data and analyzing subsequent actigraphy data for a patient who experienced relapse of depression, according to the exemplary embodiment of Example I.

FIG. 14 shows experimental data corresponding to proportion of true positive relapse patients detected over a range of time period before actual onset of depression, according to the exemplary embodiment of Example II.

FIG. 15a shows experimental data corresponding to frequency of patients that are administered self-report tests over various trigger proportions, where patient actigraphy data is used to determine when the self-report tests are administered to patients, according to the exemplary embodiment of Example III.

FIG. 15b shows experimental data corresponding to frequency of patients that are administered self-report tests over various trigger proportions, where the self-report tests are administered on a weekly basis, according to Example III.

FIG. 16a shows a subset of the experimental data of FIG. 15a for time frames where patients are in remission and not approaching relapse of depression.

FIG. 16b shows a subset of the experimental data of FIG. 15b for time frames where patients are in remission and not approaching relapse of depression.

FIG. 17a shows a subset of the experimental data of FIG. 15a for time frames where patients are approaching or experiencing relapse of depression.

FIG. 17b shows a subset of the experimental data of FIG. 15b for time frames where the patients are approaching or experiencing relapse of depression.

FIG. 18 shows experimental data for performance metrics of an exemplary method for determining relapse of depression using patient actigraphy data and self-report tests, according to the exemplary embodiment of Example III.

FIG. 19a shows data corresponding to number of relapse subjects over a course of increasing numbers of clinician visits, demonstrating a distribution of relapse visits analyzed in Example V.

FIG. 19b shows data corresponding to number of non-relapse subjects over a course of increasing number of clinician visits, demonstrating a distribution of non-relapse visits analyzed in Example V.

FIG. 20 shows exemplary timelines for three different clinical visits evaluated according to the exemplary embodiment of Example V.

### DETAILED DESCRIPTION OF THE INVENTION

The term “actigraphy” as used herein refers to a method for measuring the movement and/or activities of a patient over a period of time, and may correspond to motor activity, sleep or circadian rhythm of the patient.

The term “return” or “returned” as used herein refers to having symptoms that return after improvement and/or remission of depression within the same depression episode or return of symptoms as a new depression episode. The term “return” encompasses both relapse and recurrence of depression.

The term “relapse” or “relapsed” as used herein refers to having symptoms that return after improvement and/or remission of depression within the same depression episode. The same depression episode may be reoccurrence of depression symptoms within a predetermined time period (e.g., within the first 6 months of initiating a treatment regimen). In particular, the returned symptoms may be those that meet clinical diagnostic criteria for depression, for example, such as those clinical criteria defined in the Statistical Manual of Mental Disorders (DSM-5). There are a number of different clinical tests, in particular, those clinical tests that are administered and assessed by a clinician, that can be used to identify relapse of depression in a patient. In one example, relapse of MDD may be identified by a clinician examining a patient using the Montgomery-Åsberg Depression Rating Scale (MADRS), which is discussed further below.

The term “recurrence” as used herein refers to having symptoms that return after improvement and/or remission of depression as a new depression episode. A return of depressive symptoms after a predetermined time period (e.g., after the first 6 months of initiating a treatment regimen). The returned symptoms as a new recurrent episode of depression may be those that meet clinical diagnostic criteria for depression, for example, such as those clinical criteria defined in the Statistical Manual of Mental Disorders (DSM-5).

The term “antidepressant” as used here in refers to any pharmaceutical agent which can be used to treat depression. Suitable examples include, without limitation, a mono-amine oxidase inhibitor, tricyclic, serotonin reuptake inhibitor, serotonin noradrenergic reuptake inhibitor, noradrenergic and specific serotonergic agent, or atypical antipsychotic. Other examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, clomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citalopram, escitalopram, fluvoxamine, and the like; serotonin receptor antagonists such as nefazadone, and the like; serotonin noradrenergic reuptake inhibitors such as venlafaxine, milnacipran, desvenlafaxine, duloxetine, levomilnacipran and the like; noradrenergic and specific serotonergic agents such as mirtazapine, and the like; noradrenaline reuptake inhibitors such as reboxetine, edivoxetine and the like; atypical antipsychotics such as bupropion, and the like; natural products such as Kava-Kava, St. John's Wort, and the like; dietary supplements such as s-adenosylmethionine, and the like; and neuropeptides such as thyrotropin-releasing hormone and the like; compounds targeting neuropeptide receptors such as neurokinin receptor antagonists and the like; and hormones such as triiodothyronine, and the like. In some embodiments, the antidepressant is imipramine, amitriptyline, desipramine, nortriptyline, doxepin, protriptyline, trimipramine, maprotiline, amoxapine, trazodone, bupropion, clomipramine, fluoxetine, duloxetine, escitalopram, citalopram, sertraline, paroxetine, fluvoxamine, nefazadone, venlafaxine, milnacipran, reboxetine, mirtazapine, phenelzine, tranylcypromine, moclobemide, Kava-Kava, St. John's Wart, s-adenosylmethionine, thyrotropin releasing hormone, a neurokinin receptor antagonist, or triiodothyronine. Preferably, the antidepressant is selected from the group consisting of fluoxetine, imipramine, bupropion, venlafaxine and sertraline.

Therapeutically effective amounts/dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference (Medical Economics Company or online at http:///www.pdrel.com) or other sources.

The present application relates to systems and methods for detecting and/or predicting return of depression in patients, using passive patient data from a patient, and data corresponding to self-report tests in a computer-implemented method. The passive patient data may comprise any suitable type of data that can be collected passively during the patient's daily activities. In particular, the passive patient data can be collected without the patient actively engaging a sensor and/or device (e.g., without the patient constantly monitoring and manually providing inputs to the sensor and/or device). For example, the passive patient data may comprise passively collected data corresponding to physical behaviors of the patient and/or data corresponding to electronic device usage of the patient. In one embodiment, systems and methods for detecting and/or predicting return of depression of the present application may utilize passive patient data comprising actigraphy data.

The systems and methods of the present application may be used for patients suffering from MDD, in particular, those patients undergoing treatment for MDD, whose symptoms are in remission. The treatment may comprise psychotherapy, brain stimulation therapy, and/or administration of an antidepressant. Specifically, the patients may be those non-treatment-resistant patients having MDD, e.g., those patients with MDD and that have received and responded to, and are continuing to respond to and receive, treatment. The patients may be those patients that have received and responded to treatment and have returned to normal under treatment. In particular, the present application relates to systems and methods for detecting and/or predicting return of symptoms of MDD in patients that have responded to, and are continuing to respond to and receive an antidepressant treatment regimen. Furthermore, the system and methods of the present application may be used for patients undergoing treatment for MDD whose symptoms are in remission but have a history of prior episodes where symptoms of depression return. Although the exemplary embodiments described herein refer to relapse of depression, it is contemplated that the present application may be used to detect and/or predict any type of return of depression symptoms including recurrence of depression, which refers to return of symptoms as a new depression episode.

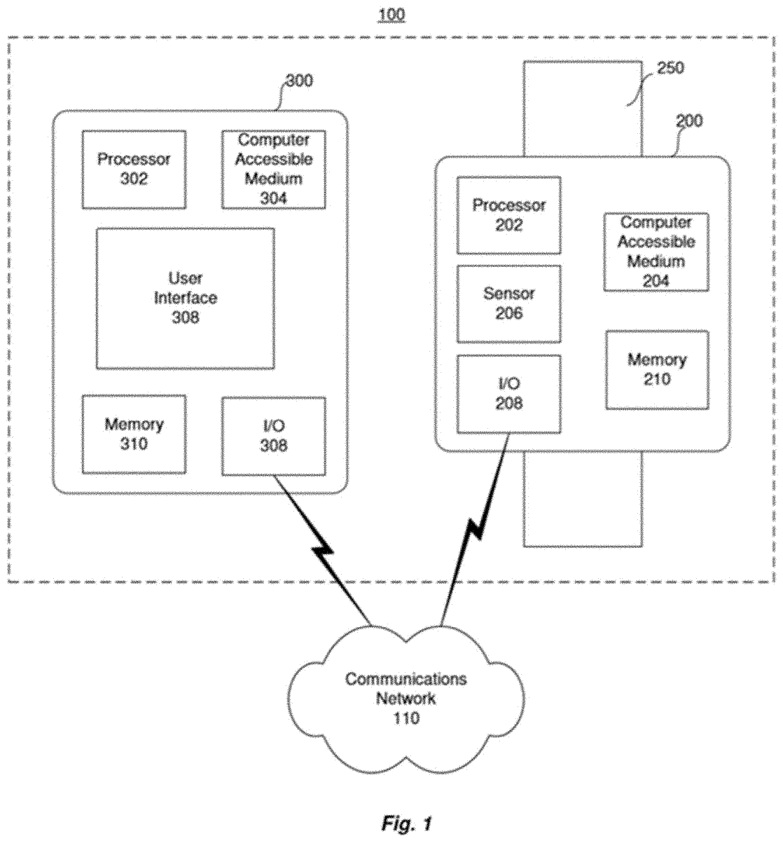

FIG. 1 shows an exemplary embodiment of a system 100 for detecting and/or predicting relapse of depression using passive patient data, and optionally, data corresponding to self-report characteristics of physical behavior (e.g., patient's self-assessment of activity or sleep adequacy). The system 100 comprises a device 200 for passively detecting and generating data corresponding to physical behaviors of the patient (e.g., physical activity, sleep, mobility, etc.) and a computing device 300 for receiving data from the device 200 and analyzing the data to determine whether the patient is likely to experience onset of relapse of depression. In one embodiment, the device 200 detects and generates actigraphy data and/or mobility data of a patient. The actigraphy data corresponding to movements of the patient over time. The mobility data corresponding to patterns of travel, such as, for example, mobility traces, by a patient over time. The device 200 is preferably suitably sized and shape to be wearable on the body of the patient. For example, the wearable device 200 may be in the form of a wearable clip that is attachable to the patient for wearing on the body of the patient throughout a day. In another embodiment, the device 200 is attached to a wearable band 250 (e.g., a watch band) for attaching the device 200 to a wrist of the patient, when the device 200 is in an operating configuration.

As shown in FIG. 1, the device 200 comprises a processor 202, a computer accessible medium 204, at least one sensor 206 and an input/output device 208. The sensors 206 may comprise actigraphy sensor(s) for detecting movements of the patient and/or mobility sensor(s) for detecting travel patterns of the patient. The actigraphy sensor may be any suitable sensor for detecting movements of the patient. For example, the actigraphy sensor may be an accelerometer for detecting movement of the patient when the device 200 is worn by the patient in an operating configuration. The mobility sensor may be any suitable sensor for detecting travel patterns of the patient. For example, the mobility sensor may be a Global Positioning System (GPS) device for detecting the positioning of the patient when the device 200 is worn by the patient in an operating configuration.

The sensor(s) 206 are operably connected to the processor 202 for providing data generated by the sensor(s) 206 to the processor 202. The processor 202 receives the data from the sensor(s) 206 to generate data corresponding to physical behaviors of the patient, such as, for example, actigraphy data and/or mobility data of the patient. The processor 202 can include, e.g., one or more microprocessors, and use instructions stored on the computer-accessible medium 204 (e.g., memory storage device). The computer-accessible medium 204 may, for example, be a non-transitory computer-accessible medium containing executable instructions therein. The system 100 may further include a memory storage device 210 provided separately from the computer accessible medium 204 for storing actigraphy data and/or mobility data therein. The input/output device 208 is any suitable device for receiving and/or transmitting data or instructions to or from the actigraphy device 200. In particular, the input/output device 208 may be a transceiver for receiving instructions to and/or transmitting data from the device 200.

The device 200 is operably connected to the computing device 300 for communicating a portion of or all of the data collected by the device 200 to the computing device 300, or for allowing the computing device 300 to retrieve a portion of or all of the data from the device 200. As shown in FIG. 1, the device 200 may be operably connected via a communications network 110 (e.g., Internet, Wi-Fi, Wide Area Network, Local Area Network, Cellular network, Personal Area Network, etc.) to the computing device 300. In particular, the input/output device 208 is operably connected to the communications network 110 to receive instructions therefrom or transmit data therethrough. In a particular embodiment, the communications network 110 is a wireless network, and more particularly, it is a short-distance wireless network, such as a Personal Area Network (e.g., Bluetooth®) having a limited range for connecting devices with a nearby proximity to the patient. However, it is also contemplated that the device 200 is directly connectable, via a wired connection, to the computing device 300.

The computing device 300 in this embodiment comprises a processor 302, a computer accessible medium 304, an input/output device 306 for receiving and/or transmitting data or instructions to or from the computing device 300. The processor 302 can include, e.g., one or more microprocessors, and use instructions stored on the computer-accessible medium 304 (e.g., memory storage device). The computer-accessible medium 304 may, for example, be a non-transitory computer-accessible medium containing executable instructions therein. The input/output device 306 may be operably connected to the communications network 110 to receive instructions therefrom or transmit data therethrough. The computing device 300 may also include a user interface 308 (e.g., a touchscreen) for obtaining input from a user and displaying an output to the user. It is contemplated that the user interface 308 may also be two separate components for displaying an output to the patient and obtaining inputs from the patient, such as, for example, a display and a keyboard. The user interface 308 is operably connected to the processor 302 to provide instructions, as discussed further below, for generating an output on the user interface and providing data corresponding to inputs obtained from the patient to the processor 302. The computing device 300 may further include a memory storage device 310 for storing past actigraphy data, past mobility data, past inputs from the patient, medical data, pharmacy data and/or at least one anomaly detector for determining a likelihood of onset of relapse of depression, the at least one anomaly detector being generated and/or trained by the computing device 300. The computing device 300 may, for example, be a mobile computing device, a smart phone, a computing tablet, a computing device, etc.

In some embodiments, the computing device 300 is also configured to collect further passive patient data, in particular, data corresponding to electronic device usage of the patient. Specifically, the computing device 300 is a mobile phone or computing tablet that the patient is also using in day-to-day activities. For example, the patient may use the computing device 300 for activities, such as web-browsing, social media use, texting, gaming, phone calls and other activities that one may typically engage with a personal electronic device. The computing device 300 may be configured to track usage of the computing device 300 during the patient's activities and generate data corresponding to usage of the computing device 300, such as, for example, keyboard use activity, keystroke dynamics, text context, etc. In this embodiment, the system 100 may collect passive patient data using both the device 200 and the computing device 300. In an alternative embodiment, the system 100 collects and analyzes passive patient data from the computing device 300, specifically, data corresponding to electronic device usage of the patient and excludes the device 200 from the system 100.

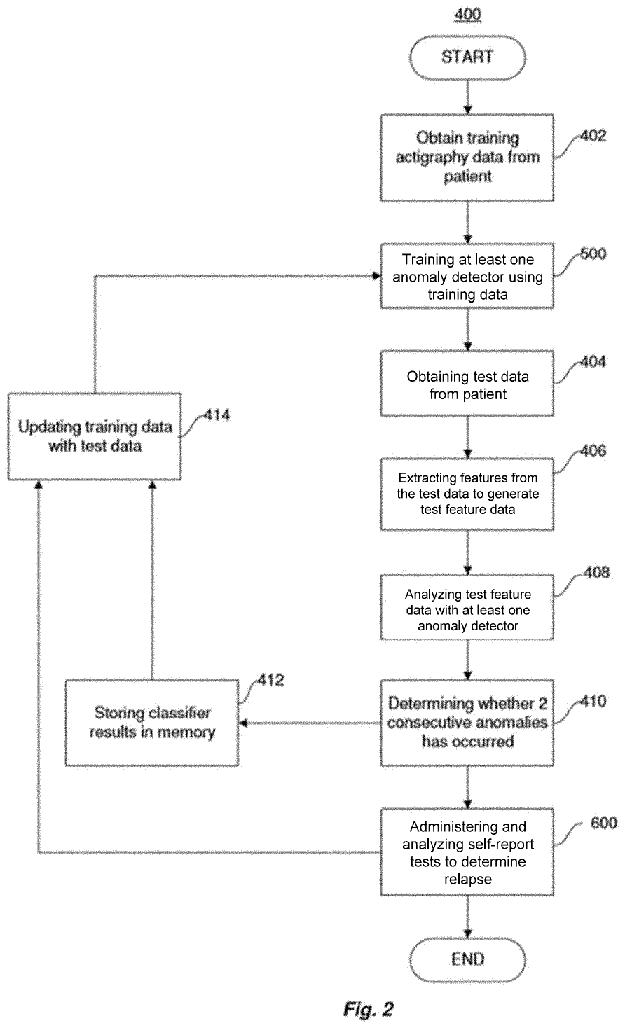

FIG. 2 shows an exemplary method 400 for detecting and/or predicting relapse of depression of a patient. The exemplary method 400 utilizes both passive patient data and administration of self-report test(s) to determine likelihood of onset of relapse of depression in a patient. The passive patient data provides for an objective and quantifiable measure that corresponds to a likelihood of onset of relapse of depression. This portion of the method 400 provides an objective trigger phase that screens the patient to determine if the passive patient data is anomalous as compared to previously collected passive patient data from the patient during a period of time when the patient is not known to have developed symptoms of depression. If the method 400 detects an anomaly in the passive patient data, then the patient may be at a higher risk of relapsing into depression. If the passive patient data suggests that the patient may be at risk, a further data may be collected in a confirmation phase of the method 400 to more precisely determine whether or not the patient is likely to be experiencing an onset of relapse of depression. In particular, the confirmation phase may include self-report test(s) conducted along quantitative scale(s) and thereby providing a further quantifiable measure corresponding to a likelihood of onset of relapse of depression.

The exemplary method 400 utilizes at least one machine-learning anomaly detector that trains the anomaly detector based on the patient's own historic data (n=1, where n is the total number of individuals sampled). Therefore, the trained anomaly detector is personalized to each individual patient. The exemplary method 400, as described below, collects training data from the patient and continues to iteratively obtain and analyze test data when the patient is not known to have relapsed into depression. The exemplary method 400 may be repeated continuously or may be iterated at desired frequencies, e.g., daily, weekly, bi-weekly, etc., therefore, allowing for regular monitoring and/or earlier detection/prediction of relapse of depression as compared to patients self-reporting and seeking medical attention only after they become aware that relapse has already occurred.

A patient's relapse into depression may be identified using one or more tests that are administered by a mental health provider (e.g., a psychiatrist, a physician, a psychologist, or a therapist) or self-administered by the patient. In this exemplary embodiment, the computing device 300 may receive medical data corresponding to the patient's medical records, such as, for example, Electronic Medical Records (EMR), and/or pharmacy data corresponding to the patient's medication records, and the computing device 300 may analyze the medical data and/or pharmacy data to determine whether the patient has relapsed into depression. If the computing device 300 determines from the medical data and/or pharmacy data that the patient has relapsed, the exemplary method 400 is terminated. However, it is contemplated that the exemplary method 400 may be restarted by manual input to the computing device 300 from a mental health provider, or upon detection by the computing device 300 from the medical data and/or the pharmacy data that the patient has returned to remission of depression.

In one exemplary embodiment, the computing device 300 may analyze the medical data and/or pharmacy data and determine that a relapse of depression has occurred when the medical data and/or pharmacy data includes data corresponding to the patient having (1) been diagnosed by a mental health provider as relapsed into depression, (2) experienced a severe symptom of depression (e.g., hospitalization for worsening of depression, suicidal ideation with intent, or suicidal behavior), or (3) scored above a predetermined threshold in a quantitative test followed by verification, as discussed further below. In particular, the quantitative test may be an evaluation by a mental health provider of the patient on the MADRS to determine whether a patient has relapsed into depression. The MADRS measures depression severity and detects changes due to antidepressant treatment. The test consists of 10 items, each of which is scored from 0 (item not present or normal) to 6 (severe or continuous presence of the symptoms), for a total possible score of 60. Higher scores represent a more severe condition. The MADRS evaluates apparent sadness, reported sadness, inner tension, sleep, appetite, concentration, lassitude, interest level, pessimistic thoughts, and suicidal thoughts.

The computing device 300 determines that the patient has relapsed when the medical data and/or pharmacy data includes data corresponding to the patient having a MADRS total score ≥22, and includes data corresponding to a subsequent verification. Data corresponding to a subsequent verification may include data corresponding to (1) a change in treatment regimen (e.g., change in type of medication, dosage of medication, or frequency of medication) within a certain period (e.g., within 14 days) from when the patient was observed as having a MADRS total score ≥22, or (2) a separate test indicating a worsening of depression. Data corresponding to the separate test may include data corresponding to an increase of at least a predetermined threshold amount on a different quantitative scale, as rated by the mental health provider. For example, the separate test may be an evaluation by a mental health provider of the patient using a Clinical Global Impression-Severity (CGI-S) scale, which is a scale for rating the severity of the patient's illness at the time of assessment, relative to the mental health provider's past experience with patients who have the same diagnosis and improvement with treatment. Considering total clinical experience, the patient is assessed on the CGI-S scale based on the severity of mental illness according to: 0=not assessed; 1=normal (not at all ill); 2=borderline mentally ill; 3=mildly ill; 4=moderately ill; 5=markedly ill; 6=severely ill; 7=among the most extremely ill patients. The computing device 300 determines that a subsequent verification has occurred when the medical data includes data corresponding to a follow-up visit to the mental health provider where the patient is evaluated as having an increase of CGI-S score by 2 or more from baseline.

In step 402, passive patient data is collected by the device 200 and/or the computing device 300 over a predetermined training time period to generate training data. The training data may comprise data corresponding to physical behaviors of the patient over the training period and/or data corresponding to electronic device usage of the patient over the training period. In one embodiment, the training data comprises training actigraphy data and/or training mobility data. Specifically, the device 200 may be worn by the patient to detect movement of the patient and generate training actigraphy data over the predetermined training time period. The training actigraphy data corresponds to movements of the patient during motor activity and/or sleep within the training period. Similarly, the device 200 may be worn by the patient to detect travel patterns of the patient and generate a set of training mobility data over the predetermined training time period.

The device 200 may be worn by the patient continuously or substantially continuously. For example, the device 200 may be worn substantially continuously such that the device is removed from the patient only for brief periods of time so as to allow the patient to engage in activities that may not be suitable or may not allow the patient to wear the device 200, such as, for example, while showering, exercising, and/or cleaning. In other embodiments, the device 200 may be worn daily by the patient. In particular, the device 200 may be worn daily by the patient during a majority of time (e.g., at least 95%, at least 90%, at least 80%, at least 70%, or at least 60% of time) that the patient is awake and/or while the patient is falling asleep or asleep. The predetermined training time period may be any suitable time period for collecting the set of training actigraphy data for training at least one anomaly detector for determining a likelihood of onset of relapse of depression. For example, the training period may be at or greater than 1 month, at or greater than 3 months, or at or greater than 6 months. In one embodiment, the training period is 3 months.

The training actigraphy data and/or the training mobility data may be stored in the memory storage device 210 of the device 200 until it is operably connected to the computing device 300 for transmitting all or a portion of the training actigraphy data and/or training mobility data to the computing device 300. Specifically, all or a portion of the training actigraphy data and/or the training mobility data may be transmitted from the device 200 via the input/output device 208 and may be received by the computing device 300 via the input/output device 306. In an alternative embodiment, the training actigraphy data and/or training mobility data is transmitted continuously from the device 200 to the computing device 300 as the actigraphy data and/or mobility data is being collected by the device 200. More particularly, the actigraphy data and/or mobility data is wirelessly transmitted from the device 200 to the computing device 300 in real-time or substantially in real-time as the actigraphy data and/or mobility data is being collected by the device 200.

In some embodiments, the training data may further comprise data corresponding to training self-report data obtained during the predetermined training time period. The training self-report data corresponding to self-report characteristics of physical behavior, as inputted by the patient to the computing device 300, over the training period. For example, the processor 302 directs the user interface 308 to display a plurality of questions that prompt responses from the patient, and receives a plurality of inputs from the user, via the user interface 308 in response to the questions. The plurality of questions may form a self-report assessment of characteristics of physical behavior (e.g., patient's self-assessment of activity, sleep adequacy, sleep quality). More specifically, the self-report assessment includes questions directed to characteristics of physical behavior that cannot be passively measured by the device 200 or the computing device 300. For example, the self-report assessment includes questions directed to a patient's perception of rest and/or sleep. In one example, the self-report assessment may include all or a portion of questions from the Medical Outcome Study Sleep (MOS-S) Scale assessment. Preferably, the self-report assessment contains a limited number of questions so as to minimize burden to the patient for active engagement (e.g., answering questions) with the user interface 308. For example, the self-report assessment may comprise no more than 12 questions, no more than 10 questions, no more than 5 questions, or no more than 3 questions. In one embodiment, the self-report assessment comprises two questions. For example, the self-report assessment comprises the two questions asking the patient to provide a quantitative assessment of perception of sleep, for example: (1) do you feel rested; and (2) do you feel you have gotten enough sleep. The self-report assessment may be repeated at any desired time intervals (e.g., daily, every other day, weekly, etc.) during the predetermined training time period and the inputs obtained during the training period are used to generate training self-report data.

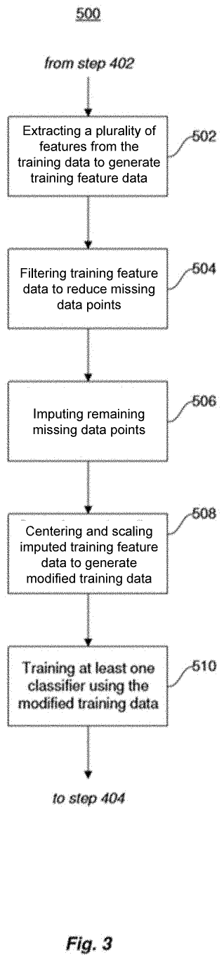

The training data obtained in step 402 is used to train at least one anomaly detector for determining a likelihood of onset of relapse of depression. The anomaly detector(s) comprise machine-learning anomaly detector(s) configured to identify deviations from the training data, which are discussed further below with respect to step 408. FIG. 3 shows an exemplary embodiment of a method 500 for training at least one anomaly detector for determining a likelihood of onset of relapse of depression using the training data obtained in step 402. In one exemplary embodiment, the training data may be transmitted to the computing device 300 and used by the computing device 300 in the exemplary method 500 to train at least one anomaly detector for determining a likelihood of onset of relapse of depression.

In step 502, the computing device 300, in particular, the processor 302, analyzes and extracts a plurality of features from the training data from step 402 to generate training feature data. In one embodiment, the processor 302 analyzes raw data obtained from the device 300 from step 402 and extracts a plurality of features from the raw data. For example, the raw data may be raw accelerometer data obtained from the device 200. In one exemplary embodiment, the processor 302 analyzes the training data from step 402 to extract a plurality of features to generate training feature data. The features may correspond to actigraphy, mobility and/or social activity of the patient. For example, the features may correspond to metrics for at least one of sleep changes, diminished ability to concentrate, diminished interested or pleasure in activities and/or depressed mood and fatigue or loss of energy, such as sleep duration, sleep start, sleep end, sleep disturbance, stationary time, phone unlock duration, phone unlock duration while in certain locations, conversation duration, number of places visited, time spent in certain locations, heart rate, fractal activity patterns, monofractal patterns or multifractal dynamics of activity data, entropy of all or a portion of activity data, among others such as those described in Wang et al., “Tracking Depression Dynamics in College Students Using Mobile Phone and Wearable Sensing,” Proceedings of the ACM on Interactive Mobile Wearable and Ubiquitous Technologies, Vol. 2, No. 1, Article 43 (March 2018), which is incorporated by reference in its entirety herein.

In particular, the processor 302 analyzes and extracts a plurality of actigraphy features from the training actigraphy data from step 402 to generate at least a portion of the training feature data. The extracted actigraphy features may correspond to metrics for at least one of motor activity, sleep, wakefulness and circadian rhythm of the patient. For example, the features may comprise metrics for sleep duration (e.g., total hours of sleep time per night), sleep pattern (e.g., time of day for sleep onset, time of day for sleep end), sleep quality (e.g., mean activity counts per minute during rest period, percentage of sleep fragmentation, percentage of sleep efficiency, actigraphic estimate of minutes of wake after sleep onset), fractal patterns or dynamics/behaviors during activity or sleep (e.g., changes to monofractal patterns or multifractal dynamics during active or sleep hours, temporally local fluctuations to sleep patterns captured by different scaling properties at different times of activity), daytime activity (e.g., mean daytime activity counts per minute, peak daytime activity counts per minute), and entropy during activity or sleep (e.g., measurements of randomness and chaos signatures in activity or during sleep). More particularly, the actigraphy features include metrics for detecting sleep disturbance, which may include metrics for sleep, wakefulness and/or circadian rhythm of the patient. It is believed that sleep disturbance is a common symptom in patients with MDD and patients often experience reduced sleep quality prior to or during recurrence or relapse of a depressive episode.

Entropy of the actigraphy data provides a quantified metric for complexity of the data, which may be used as one of the plurality of actigraphy features. In one example, the actigraphy data for an activity time series may be represented as x(i), i=1 to N with Δt=1 minute. Sample entropy (SaEn) of the activity time series x(i) involves reconstruction of vectors from the activity time series that are (m-dimensional) state space representations of the dynamics of an overall system from which the actigraphy data is obtained. The vectors may be represented as V(i)={x(i), x(i+δ), . . . , x(i+[m−1]δ}, where δ is a time delay between the successive components of the vector. The vectors in this example are set to unity in complexity analysis. SaEn is determined as the logarithmic difference between the probability (density) of occurrence of vector V(i) within a chosen distance r in m dimension and the probability of occurrence of vector V(i) within the same chosen distance r in m+1 dimension. The densities of state space ρ^{m}(r) and ρ^{m+1}(r) are measures of the fractions of reconstructed vectors that fall within a chosen radius r in m dimension and in m+1 dimension, respectively. SaEn may be represented as:

SaEn(m,r)=log [ρ^{m}(r)/ρ^{m+1}(r)]

In one exemplary embodiment, each day of actigraphy data may be partitioned evenly into four epochs: morning (6 am to 12 pm), afternoon (12 pm to 6 pm), evening (6 pm to 12 am), and night (12 am to 6 am). SaEn may be determined for each day and for each epoch. In one example, SaEn is determined as the median SaEn value (on z-score activity counts) across the last 7 days of activity counts (without any identified gaps).

The features may comprise one or more of the exemplary actigraphy features listed and defined below in Table 1.

TABLE 1

| Actigraphy Features | Definition |
|---|---|
| Active Spectral Entropy | Spectral content as measured by entropy |
| | during awake period |
| Active Lempel Ziv | Spectral content as measured by signal |
| Complexity | complexity during awake period |
| Active Percentage | Percentage of sleep duration during awake |
| of Sleep Duration | period |
| Active Average Duration | Duration of wake bouts within a sleep |
| of Wake Bouts Min. | epoch during awake period |
| Active Sleep Fragment- | Sleep fragmentation (disruption) during |
| ation Index | awake period |
| Active Total Sleep | Total sleep time in minutes during awake |
| Time Min. | period |
| Active Total Wake | Total wake time within a sleep epoch in |
| Time Min. | minutes during awake period |
| Active Number of | Total number of wake bouts within a sleep |
| Wake Bouts | epoch during awake period |
| Sample Entropy During | Spectral content as measured by sample |
| 6AM to 12PM | entropy during 6 AM to 12 PM |
| Sample Entropy During | Spectral content as measured by sample |
| 12PM to 6PM | entropy during 12 PM to 6 PM |
| Sample Entropy During | Spectral content as measured by sample |
| 6PM to 12AM | entropy during 6 PM to 12 PM |
| Sample Entropy During | Spectral content as measured by sample |
| 12AM to 6AM | entropy during 12 AM to 6 AM |
| Sleep Total Activity | Total activity during sleep period |
| Sleep Average Activity | Average activity per minute (cycles per |
| Per Minute Cpm. | minute) during sleep period |
| Sleep Maximum Activity | Maximum activity during sleep period |
| Sleep Spectral Entropy | Spectral content as measured by entropy |
| | during sleep period |
| Sleep Lempel Ziv | Spectral content as measured by signal |
| Complexity | complexity during sleep period |
| Sleep Percentage of | Percentage of sleep duration during sleep |
| Sleep Duration | period |
| Sleep Duration Between | Sleep duration between end of sleep and |
| End of Sleep and End | end of rest in minutes |
| of Rest Min. | |
| Sleep Hours From Noon | Hours from noon for rest onset |
| For Rest Onset Hours | |
| Sleep Average Duration | Average duration of wake bouts in |
| of Wake Bouts Min. | minutes during sleep period |
| Sleep Efficiency | Scored total sleep time of the sleep period |
| | divided by total time in bed minus total |
| | invalid time (Sleep/Wake) multiplied |
| | by 100 |
| Sleep Fragmentation | The sum of Percent Mobile and Percent |
| Index | Immobile Bouts Less Than 1-Minute |
| | Duration to the Number of Immobile |
| | Bouts for a given interval Percent |
| | Immobile Bouts = Scored Total Immobile |
| | Time divided by (Interval Duration minus |
| | Total Invalid Time (Activity)) |
| | multiplied by 100 Percent Mobile |
| | Bouts = Scored Total Mobile Time |
| | divided by (Interval Duration minus Total |
| | Invalid Time (Activity)) multiplied by 100. |
| | Number of Immobile Bouts = Total number |
| | of continuous blocks, one or more epochs in |
| | duration, with each epoch of each block |
| | scored as Immobile, between the Start Time |
| | and the End Time of the given interval |
| Sleep Onset Latency | The time elapsed between the Start Time of |
| Min. | a given Rest Interval and the following |
| | Sleep Start Time, in minutes |
| Sleep Hours From Noon | Hours from Noon for sleep onset |
| For Sleep Onset Hours. | |
| Sleep Wake After Sleep | Duration of wake episode in minutes after |
| Onset Min. | sleep onset during sleep period |
| Sleep Total Sleep | Duration of total number of sleep episodes |
| Time Min. | in minutes during sleep period |
| Sleep Total Wake | Duration of total number of awake episodes |
| Time Min. | in minutes during sleep period |
| Sleep Valid Rest | Duration of total rest duration minus |
| Duration Min. | unscored (invalid) sleep epochs |
| Sleep Valid Sleep | Duration of total sleep duration minus |
| Duration Min. | unscored (invalid) sleep epochs |
| Sleep Number of | Number of wake bouts during sleep period |
| Wake Bouts | |

In addition, the actigraphy data can include fractal fluctuations (e.g., temporal, structural and/or statistical fluctuations at a wide range of time scales), which is believed to be stable within the same individuals but can be sensitive to pathological conditions. Data demonstrating fractal regulation is believed to represent a physiological system's adaptability and to reflect the complexity of physiological networks in which regulatory processes function interactively over a wide range of time scales. Therefore, measures of fractal fluctuations of the actigraphy data may be extract as actigraphy features in lieu of or in conjunction with one or more of the features identified above. In one example, the actigraphy data displays complex temporal fluctuations characterized by scale-invariant (monofractal) patterns which may be utilized as actigraphy features. The monofractal patterns are homogenous and have the same scaling properties throughout the entire signal. The actigraphy data can also exhibit a special class of complex process called multifractal which may also be included as actigraphy features. Multifractal behavior is characterized by distinct signatures at different time scales (minutes to hours). The multifractal behavior may include multiple co-existent dynamic processes that may generate temporally local fluctuations captured by different scaling properties at different times.

Fractal patterns of the actigraphy data may be determined using detrended fluctuation analysis (DFA) methods. The DFA methods determine the scaling behavior of fluctuations within the actigraphy data over a range of time scales from minutes to hours. DFA methods examine multiscale correlations of activity fluctuations at multiple time scales. For example, a DFA method provides a fluctuation amplitude F(n) as a function of time scales n. For long range-correlated data F(n) follows a power law F(n)˜n^{α}, where scaling or fluctuation exponent (α) quantifies the multiscale correlations as follows: if α=0.5, there is no correlation in the fluctuation (“white noise”); if α>0.5, there is positive correlations (large values are more likely to be followed by large values (and vice versa)) in the fluctuation; if α<0.5, there is negative correlations (large values are more likely to be followed by small values (and vice versa)) in the fluctuation. Many physiological outputs under healthy conditions exhibit fluctuation exponent (a) values close 1.0 indicating the most complex underlying control mechanisms. DFA use a second order polynomial function to detrend data to eliminate the effect of possible linear trends in the data.

In one embodiment, the multifractal dynamics of the actigraphy data is determined using a MFDFA method. In the exemplary MFDFA method of the present application, fluctuation of the data is generally represented as:

$${F}_{q} ( s ) = {〈 {\{ {\sum }_{j = 1}^{s} {( {Y}_{v} ( j ) - {p}_{s , v}^{k} ( j ) )}^{2} \}}^{\frac{q}{2}} 〉}^{\frac{1}{q}}$$

where Y is the profile function created as a cumulative sum of data (mean subtracted) and is divided into nonoverlapping subsequences of ν each of length s. The data in each segment ν is fitted by k-th order polynomial p^{k}, and q is an index variable indicating a qth order fluctuation. The generalized representation of fluctuation under the MFDFA method simplifies to a DFA method when q=2. In one example, k=2 indicating that a second order polynomial is used in the MFDFA method. The fluctuation function F_{q} (s) simplifies to a monofractal detrended fluctuation analysis when q=2. For multifractal signals, F_{q}(s) follows a power law: F_{q}(s)˜s^{h(q)}, where h(q) is defined as a generalized Hurst exponent. Further, for a multifractal signal, h(q) varies nonlinearly with q according to the equation τ(q)=qh(q)−1. One way to characterize the multifractal time series is by the singularity spectrum f(α) which can be related to τ(q) by a Legendre transform as follows: f(α)=qα−τ(q) where α=h(q)+qh′(q).

In one example, the DFA method may be a particular embodiment of the MFDFA method where the MFDFA method may derive amplitude fluctuations F(n) at different time scales n, represented as F(n)≈n^{α}, with the scaling or fluctuation exponent α indicating correlations in fluctuations. A value of α>0.5 indicates positive correlation (large values are more likely to be followed by large values (and vice versa)), α<0.5 indicates negative correlation (large values are more likely to be followed by small values (and vice versa)) and α=1 indicates long range correlation or monofractal representation. Many physiological outputs under healthy conditions exhibit α values close to 1.0, indicating the most complex underlying control mechanisms. The amplitude fluctuations are quantified with different moments. A multifractal spectrum is then computed as a distribution of different scaling exponent α with respect to the different moments. A broader width (αₘₐₓ−αₘᵢₙ) in the multifractal spectrum indicates presence of multifractal dynamics and shorter width indicates loss of multifractal dynamics or presence of monofractal dynamics. Multifractal spectrum represents two measurable dimensions, the Dq (q-order singularity/fractal dimension) and the hq (q-order singularity exponent) (as mentioned in Matic et al., “Objective differentiation of neonatal EEG background grades using detrended fluctuation analysis.” Front. Hum. Neurosci., 9: 189 (2015) (available at https://www.frontiersin.org/articles/10.3389/fnhum.2015.00189) and incorporated by reference herein). In the visual assessment of multifractal spectra, differences can be noticed in a horizontal and vertical positioning (hq, Dq values), a width (width_hq), as well as in the general shape of the multifractal spectra reflecting temporal variations of local Hurst exponents and these features could be used as a plurality of actigraphy features for training an anomaly detector. Exemplary multifractal detrended fluctuation analysis methods are described in Ihlen et al., “Introduction to multifractal detrended fluctuation analysis in Matlab,” Front. Physiol., Vol. 3, Article 141, 1-18 (available at https://www.frontiersin.org/articles/10.3389/fphys.2012.00141/full), Ivanov, et al. “Multifractality in human heartbeat dynamics.” Nature 399: 461-465 (1999) (available at https://www.nature.com/articles/20924), Franca et al., “On multifractals: a non-linear study of actigraphy data.” Physica A: Statistical Mechanics and its Applications 514: 612-619 (2019) (available at https://www.sciencedirect.com/science/article/pii/S037843711831255X), and Kantelhardt et al., “Multifractal detrended fluctuation analysis of nonstationary time series,” Physica A, 316: 87-114 (2002), all of which are incorporated by reference herein.

Other features, in lieu of or in conjunction with, the actigraphy features described above may be extracted from the training data. For example, features may be extracted from mobility data of a patient such as for example, mobility traces, geographical features, total distance covered, maximum distance between two locations, radius of gyration, standard deviation of displacements, maximum distance from home, number of different places visited, number of different significant places visited, routine index, such as those described in Barnett et al., “Inferring Mobility Measure from GPS Traces with Missing Data,” Biostatistics, pp. 1-33 (2018); Canzian et al., “Trajectory of Depression: Unobstrusive Monitoring of Depressive States by means of Smartphone Mobility Traces Analysis” UbiComp '15: Proceedings of the 2015 ACM International Joint Conference on Pervasive and Ubiquitous Computing, 1293-1304 (September 2015); and Canzian et al., “From Mobile Phone Monitoring of Depressive States using GPS Traces Analysis to Data-Driven Behavior Change Interventions,” Frontiers in Public Health (January 2016), which are incorporated by reference herein in their entirety. In another example, additional features may be extracted data corresponding to electronic device usage of the patient, such as those described in Mastoras et al., “Touchscreen typing pattern analysis for remote detection of the depression tendency,” Nature Scientific Reports, 9:13414 (2019) and Zulueta et al., “Predicting Mood Disturbance Severity with Mobile Phone Keystroke Metadata: A BiAffected Digital Phenotyping Study,” J. Med. Internet Res., 20(7):e241 (July 2018), which are incorporated by reference herein in their entirety.

The processor 302 may extract from the training data any suitable number of features. An increased number of features may improve predictive performance of the systems and methods of the present application but may become computationally burdensome. Therefore, an appropriate number of features may be selected to balance predictive performance with computational efficiency. In some embodiments, the processor 302 may extract from the training data at least 10, at least 20, at least 30, at least 40, or at least 50 different features. In one particular embodiment, the processor 302 extracts 31 features from the training actigraphy data. More particularly, the processor 302 may extract some or all of the features identified above in Table 1. In another embodiment, the processor 302 may extract from the training data features for detecting sleep disturbance which may include metrics for sleep, wakefulness and/or circadian rhythm of the patient. In a further embodiment, the processor 302 may extract from the training data features including monofractal patterns during activity or sleep, multifractal dynamics/behaviors during activity or sleep, and/or entropy during activity or sleep.

In step 504, the processor 302 filters the training feature data extracted from the training data such that features and/or time points, where a significant portion (e.g., more than 30%, more than 40%, or more than 50%) of data points is absent, are removed from the training feature data to generated filtered training data. For example, the filtered feature data retains only those features where less than 40% of the data points for that feature are missing, and only those time points where less than 40% of the features for that time point are missing.

In step 506, the processor 302 may further modify the filtered training data by imputing missing data points to generate imputed training data. In one exemplary embodiment, missing data points may be estimated by the processor 302 by analyzing a plurality of its nearest neighbors to generate an estimated value for the missing data points based on the nearest neighbors. For example, features missing data at certain time points may be estimated by the processor 302 using data from time points adjacent to those time points where data are absent. Each of the missing data points may be estimated using its k nearest neighbors, where k may be from 2 to 10, from 3 to 8, or from 4 to 6. In one embodiment, k=5. The processor 302 may use any suitable method to estimate the missing data points based on its k nearest neighbors. For example, the processor 302 may utilize a Euclidean distance, based on multi-parameters from the training data, and isolate the closest k neighbors and use the mean values of the different features as inputs for the missing features. In another exemplary embodiment, missing data points may be estimated by the processor 302 by analyzing the filtered training data using bagged trees. Specifically, for each feature, a decision tree module, in particular, a bagged tree module, may be created using the remaining other features of the filtered training data to impute missing data. Furthermore, the decision tree module may include surrogate splits such that the decision tree module traces to a left or right child node, using the best surrogate predictor, to impute the missing data points. In step 508, the imputed training data may be centered and scaled using population mean and standard deviation (data-mean (population data)/standard deviation (population data). A power transformation (e.g., a YeoJohnson transformation, or a Box Cox transformation) may also be applied to all features of the imputed training data to generate modified training data having a normalized distribution.

The modified training data obtained from step 508 is used by the processor 302 in step 510 to train at least one anomaly detector for determining a likelihood of onset of relapse of depression. Once the anomaly detector(s) are trained by the processor 302 according to the exemplary method 500 shown in FIG. 3, the method 400 proceeds to step 404 to obtain test data from the patient. Similar to step 402, in step 404, test data is collected by the device 200 and/or the computing device 300 after the training period, and therefore, test data obtained in step 402 is data that was not previously utilized in training the anomaly detector in method 500. The test data may be generated in real-time or may be generated over a desired test period. The test period may be for a duration from at or about 1 day to at or about 2 weeks. In one embodiment, the test period is at or about 1 week or at or about 2 weeks. In step 406, the processor 302 analyzes and extracts a plurality of features from the test data in a similar manner as discussed above with respect to step 502 to generate test feature data.

In step 408, the processor 302 analyzes the test feature data from step 406 using the anomaly detector(s) trained by method 500, specifically, in step 510, to determine a likelihood of onset of relapse of depression. In particular, the anomaly detector(s) compare the test feature data to the training data (or data derived from the training data, such as the modified training data) to determine a likelihood of onset of relapse of depression. In one embodiment, the processor 302 may analyze the test feature data using the anomaly detector(s) to determine whether the test feature data is likely an anomaly as compared to the training data. The anomaly is data having different characteristics from the training data. As discussed above, the training data is collected when the patient has not experienced onset of relapse of depression, and therefore, an anomaly from the training data corresponds to an onset of relapse of depression.

The anomaly detector(s) comprise machine-learning anomaly detector(s) configured to identify deviations from the training data. The anomaly detector(s) may comprise supervised and/or unsupervised learning anomaly detectors. For example, the anomaly detector(s) may construct profiles of normal instances using the modified training data (e.g., when the patient has not relapsed into depression) and identify as anomalies (e.g., when the patient experiences onset of relapses of depression) any further data that deviate from the normal profiles. In an alternative, the anomaly detector(s) may identify as anomalies by isolating any further data that deviate from the modified training data using a plurality of binary trees.

The anomaly detector(s) may include any suitable type of anomaly detector(s) for detecting an anomaly from the modified training data. For example, the anomaly detector(s) may utilize one-class support vector machines (one-class SVMs), isolation forest (IF) modules, one-class neural network (e.g., Long short-term memory (LSTM) network), and other one-class analysis methods. In one embodiment, the anomaly detector(s) comprise at least one of a one class SVM and IF binary trees. Exemplary one-class SVMs that may be applied to the actigraphy data of the present application include those describe by Scholkopf et al., “Support Vector Method for Novelty Detection,” Advances in Neural Information Processing Systems, 582-588 (2000), Tax & Duin, “Support Vector Data Description,” Machine Learning, 54:45-66 (2004) and Manevitz and Yousef, “One-Class SVMs for Document Classification,” Journal of Machine Learning Research, 2: 139-154 (2001), all of which are incorporated by reference in their entirety herein. In one exemplary embodiment, the anomaly detector(s) comprise the outlier SVM, as described in Manevitz and Yousef, “One-Class SVMs for Document Classification,” Journal of Machine Learning Research, 2: 139-154 (2001), which is incorporated by reference herein. As another example, an exemplary IF anomaly detector, using IF binary trees, may be applied to the actigraphy data of the present application. The IF anomaly detector include the iForest methodology described in Liu et al., “Isolation-Based Anomaly Detection,” ACM Transactions on Knowledge Discovery from Data, 6(1): 1-39 (March 2012), which is also incorporated by reference herein.

In one embodiment, the processor 302 analyzes the test feature data using the at least one anomaly detector to generate a binary output (i.e., 0 or 1 indicating whether or not the test data likely corresponds to a relapse of depression) and/or to generate an anomaly score corresponding to a probability that the test data likely corresponds to a relapse of depression. In particular, the anomaly detector(s) may comprise a one-class SVM anomaly detector configured to generate a binary output, where 0 indicates that the test feature data likely corresponds to non-relapse of the patient and 1 indicates that the test feature data is likely an anomaly compared to the training data. The anomaly detector(s) may further comprise, or in the alternative, comprise a tree-based anomaly detector, specifically an IF anomaly detector, to generate an anomaly score corresponding to a probability that the test feature data is likely an anomaly. The processor 302 determines that the test feature data is likely an anomaly compared to the training data when the anomaly score is above a certain threshold. The threshold value may be selected so as to discern signal vs. noise—separating those data points that are likely to correspond to an anomaly from data points corresponding to general variability in the patient's test data as analyzed by the IF anomaly detector. For example, the processor 302 may determine that the test feature data is likely to correspond to an anomaly, and therefore, likely to correspond to a relapse of depression when the anomaly score from the IF anomaly detector is ≥0.6.

In one embodiment, the anomaly detector may model normal behavior of the actigraphy data and use prediction errors of the model to identify anomalies. For example, the anomaly detector may utilize Long Short Term Memory (LSTM) neural networks to analyze multifractal dynamics of test actigraphy data to quantify prediction errors, which is subsequently used to identifying anomalies. More specifically, the anomaly detector analyzes actigraphy data via stacked LSTM neural networks having two components: an encoder that learns vector representation of an input time-series and a decoder that uses the vector representation to reconstruct the time-series. The reconstruction error of test feature data is used to determine the likelihood of anomaly.

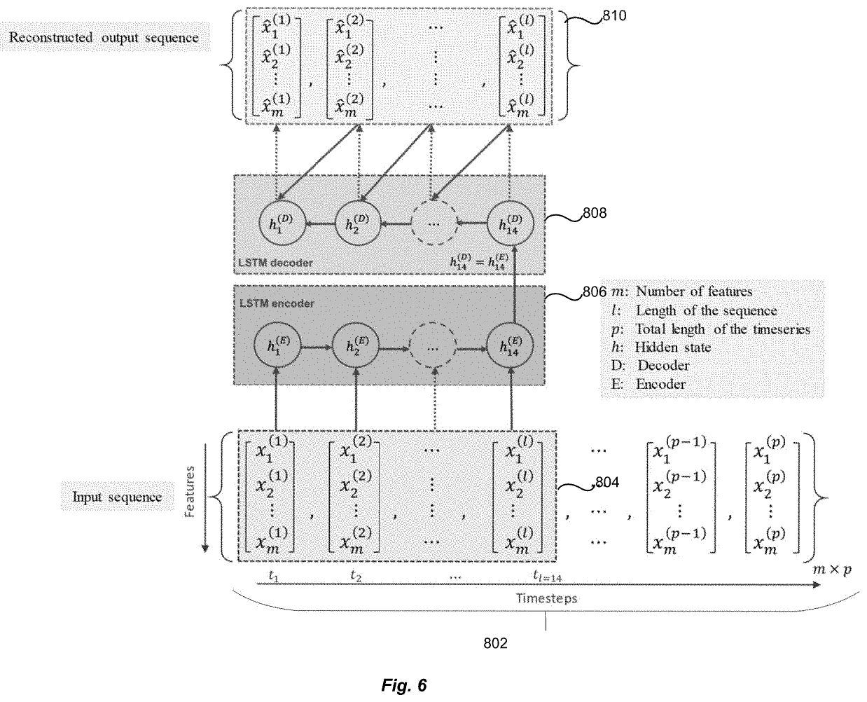

FIG. 6 shows an exemplary a schematic of a LSTM anomaly detector having an encoder and a decoder (Enc-Dec AD). The actigraphy feature data 802 extracted from actigraphy data collected from a patient over time is represented as a time series of vector over time, where t indicates the earliest time point in the data series. In FIG. 6, the actigraphy feature data 802 is extracted for m number of features from actigraphy data collected for a period of time, having a total length of p. A subset of the actigraphy feature data 804, data for a time sequence having a length of l, may be the test feature data. The Enc-Dec AD comprises an encoder 804 for learning the time sequence of the subset of actigraphy feature data 806, using the results from the encoder 604 to then reconstruct an output sequence 810 using a decoder 808. The Enc-Dec AD determines the error vectors for each point in the actigraphy actigraphy feature data. The error vector for time point tᵢ is determined as e⁽ⁱ⁾=|x⁽ⁱ⁾−\hat{x}⁽ⁱ⁾|, where x⁽ⁱ⁾ is the empirically observed value at time point tᵢ and \hat{x}⁽ⁱ⁾ is a reconstructed value at time point tᵢ determined by the Enc-Dec AD. Error vectors generated from training actigraphy data, which is collected during which the patient is not known to have developed symptoms of depression, are used to determine the mean (μ) and the standard deviation (Σ) of a Normal distribution [symbol] (μ, Σ) using Maximum Likelihood Estimation. An anomaly score, or Mahalanobis distance, a⁽ⁱ⁾=(e⁽ⁱ⁾−μ)^{T}Σ⁻¹(e⁽ⁱ⁾−μ) is computed from the error vectors generated based on the μ and Σ of the error vectors generated from the training actigraphy data.

Because relapse into depression typically occurs gradually over weeks, persistent detection of actigraphy markers for potential relapse of depression over a period of time can be useful for improving specificity of and reduce likelihood of false positives generated by the exemplary method 400. Therefore, the processor 302 may analyze a plurality of results generated by the anomaly detector(s) over a desired period of time, such as, for example, at least 1 week, at least 2 weeks, or at least 1 month. Result(s) from analyzing the prior test data using the anomaly detector(s) in a prior iteration of steps 404 to 408 may be stored in the memory storage device 310, as will be discussed further below. In step 410, the processor 302 analyzes the results from the current iteration of step 408 and any available prior data stored in memory storage device 310 corresponding to results generated by the anomaly detector(s) using prior test data in earlier iterations of step 408. In particular, step 410 analyzes the result(s) from step 408 and prior data stored in the memory storage device 310 to determine whether the anomaly detector(s) have persistently identified test feature data as likely anomalies over a period of 1 week or over a period of 2 weeks. In one exemplary embodiment, the method 400 is iterated weekly and step 410 analyzes the result(s) generated from step 408 and prior data stored in memory storage device 310 to determine whether the anomaly detector(s) have persistently identified likely anomalies in two consecutive iterations of the method 400.

If the processor 302 in step 410 determines that the patient's test feature data has not persistently been identified by the anomaly detector(s) as likely anomalies for the desired period of time, then the method proceeds to step 412 and stores the result(s) from step 408 in memory storage device 310 to be used as results generated using prior test data in the next iteration of the method 400. If the processor 302 in step 410 determines that the patient's test feature data has persistently been identified by the anomaly detector(s) as likely corresponding to anomalies for the desired period of time, then the method 400 proceeds to method 600 to administer at least one self-report test via the computing device 300, more particularly, the processor 302 automatically proceeds to method 600 to administer the self-report test(s) via the computing device 300 to the patient and further determine whether the patient is likely to experienced onset of a relapse of depression based on the self-report test(s).

Typically, the self-report test(s) require the patient to actively engage with the computing device 300 to answer a series of survey questions. The exemplary method 400 imposes a lower burden on the patient because passive patient data is passively collected from the patient (e.g., by wearing the actigraphy device 200) and self-report test(s) are administered only after the anomaly detector(s) have identified anomalies using actigraphy data in step 410. Therefore, method 400 administers self-report test(s) at a lower frequency as compared to such tests being administered at a regular (e.g., weekly) schedule. The analysis of step 410 allows the processor 302 to use actigraphy data that are passively collected from the patient for sensitivity in detection of relapse of depression. The additional steps for administering self-report test(s) provide further specificity in prediction and/or detection of relapse of depression, but are less frequently administered thereby reducing burden to the patient for active engagement (e.g., answering survey questions) with the computing device 300. The lowered burden to the patient promotes patient comfort and compliance with the exemplary method 400.

FIG. 4 shows an exemplary method 600 for administering at least one self-report assessment test via the computing device 300 and further determining whether the patient is likely to experience onset of relapse of depression. In one exemplary embodiment, the method 400 is repeated on a weekly basis. In step 602, the computing device 300 actively engages with the patient by administering self-report test(s) to the patient. Specifically, the processor 302 directs the user interface 308 to display a plurality of survey questions that prompt responses from the patient, and receives a plurality of inputs from the user, via the user interface 308, in response to the series of survey questions. The self-report test(s) may comprise any suitable test having a series of survey questions corresponding to symptoms of depression for prompting the patient to input a series of responses along a quantitative scale (e.g., rating on a numerical scale for each symptom). For example, the self-report test(s) may comprise survey questions for prompting the patient to input a series of responses along a quantitative scale for assessing MDD symptoms, anxiety symptoms, sleep disturbance, anhedonia, energy/motivation, antidepressant medication, adherence, functioning/disability, health-related quality of life, pain, self-insights regarding the first sign that may precede relapse, healthcare utilization, and/or stress/resilience. For example, the self-report test(s) may comprise evaluations using Pain Frequency, Intensity and Burden Scale (P-FIBS), Health Resource Use Questionnaire (HRUQ), Recent Life Changes Stress Test (RLCST), Perceived Stress Scale (PSS), Snaith Hamilton Pleasure Scale (SHAPS), WHO Disability Assessment Schedule (WHODAS 2.0), EuroQol health state in 5 dimensions and 5 levels (EQ-5D-5L), General Anxiety Disorder 7-Item Scale (GAD-7), Sleep measure (MOS Sleep-R), Patient Adherence to Antidepressant Medication Questionnaire (PAQ), Quick Inventory of Depressive Symptoms (QIDS-SR16), Very Quick Inventory of Depressive Symptoms (VQIDS-SR5), Rothschild Scale for Antidepressant Tachyphylaxis (R-SAT), etc.

In one embodiment, the self-report test(s) comprise evaluations using QIDS-SR16 and/or GAD-7. The QIDS-SR16 is a patient-reported measure designed to assess the severity of depressive symptoms. The QIDS-SR16 assesses all the criterion symptom domains designated by the DSM-5 to diagnose a major depressive episode. Patients provide responses to each of 16 items with a 4-point scale, with scores ranging from 0 to 3 for each item. The scoring system of the QIDS-SR16 converts responses to the 16 separate items into the 9 DSM-5 symptom criterion domains comprising: 1) sad mood; 2) concentration; 3) self-criticism; 4) suicidal ideation; 5) interest; 6) energy/fatigue; 7) sleep disturbance (initial, middle, and late insomnia or hypersomnia); 8) decrease or increase in appetite or weight; and 9) psychomotor agitation or retardation. The total score is obtained by adding the scores for each of the 9 symptom domains of the DSM-5 MDD criteria: 4 items are used to rate sleep disturbance (early, middle, and late insomnia plus hypersomnia); 2 items are used to rate psychomotor agitation and retardation; 4 items are used to rate appetite (increase or decrease and weight increase or decrease). One item is used to rate the remaining 6 domains (sad mood, interest, energy/fatigue, self-criticism, concentration, and suicidal ideation). Using a scale of severity of depression of none, mild, moderate, severe, and very severe, corresponding QIDS-SR16 total scores are none, 1 to 5; mild, 6 to 10; moderate, 11 to 15; severe, 16 to 20; and very severe, 21 to 27. The GAD-7 is a 7-item self-report assessment of anxiety. Each item is scored on a 4-point scale (0 to 3), with a total score range of 0 to 21. A GAD-7 score ≥5 correlates to mild depression. A GAD-7 score ≥10 correlates to moderate to severe depression.

In step 603, the processor 302 analyzes data corresponding to the plurality of inputs from the patient in response to the plurality of survey questions of the self-report test(s) of step 602 to determine whether the patient is likely to experience onset of relapse of depression, and if so, proceed to step 610. Specifically, the processor 302 may analyze a resulting score generated based on the plurality of inputs from the patient in a self-report test, and determine that the patient is likely to experience onset of relapse of depression when the resulting score is at or above a first threshold value. For example, the first threshold value for the QIDS-SR16 score is 11. In another example, the first threshold value for the GAD-7 score is 10.

If the resulting score(s) of the self-report test(s) do not meet the first threshold values, the method 600 proceeds to further analyze data corresponding to the plurality of inputs from the patient in response to the plurality of survey questions of the self-report test(s) with prior data stored in the memory storage device 310. In step 604, the processor 302 determines whether self-report test(s) were consecutively administered to collect data of patient behavior within a most recent predetermined period using prior data stored in memory storage device 310. The predetermined period may be a two-week period or a three-week period. In one exemplary embodiment, the method 400 is repeated on a weekly basis and the processor 302, in step 604, analyzes whether prior data stored in the memory storage device 310 indicates that self-report test(s) have been administered for three consecutive iterations (including the test administered in step 602) of the method 400. If so, the method 600 proceeds to step 606. If the processor 302 determines that self-report test(s) have been administered for two consecutive iterations of the method 400 (including the test administered in step 602), then the method proceeds to step 608. More specifically, if the processor 302 determines that self-report test(s) were administered for the most recent two iterations (including the test administered in step 602) of the method 400, the method 600 proceeds to step 608. Otherwise, the method 600 proceeds to step 612.

In step 606, the processor 302 further analyzes data from step 602 and prior data stored in the memory storage device 310 to determine whether the patient is likely to experience onset of relapse of depression, and if so, proceed to step 610. Specifically, the processor 302 may analyze the resulting score generated based on the plurality of inputs from the patient in a self-report test, and determine that the patient is likely to experience onset of relapse of depression when the processor 302 determines that the current resulting score and prior data stored in the memory storage device 310 indicates that the patient's self-report test scores have been above a second threshold value for at least 2 weeks during the preceding predetermined period (e.g., most recent three-week period). The second threshold value is lower than the first threshold value. In this embodiment, the processor 302 may determine that the patient is likely to experience onset of relapse of depression (step 610) when the resulting score is above the second threshold value and the prior data in the memory storage device 310 indicates that the resulting score has increased over at least two consecutive iterations of the method 400. In addition to or alternatively, the processor 302 may determine that the patient is likely to experience onset of relapse of depression (step 610) when the processor 302 determines that the resulting score has been above the second threshold value at least once within the preceding predetermined period, and that the resulting score has increased during the preceding predetermined period (e.g., by at least 1 point increase in the resulting score), indicating worsening of the patient's depression symptoms. For example, the second threshold value for the QIDS-SR16 score is 9. In another example, the second threshold value for the GAD-7 score is 6. In a further example, the second threshold value for the GAD-7 score is 5.

Similar to step 606, the processor 302 in step 608 analyzes data from step 602 and prior data stored in the memory storage device 300 to determine whether the patient is likely to experience onset of relapse of depression and if so, proceed to step 610. In particular, the processor 302 in step 608 may analyze the resulting score generated based on the plurality of inputs from the patient in a self-report test, and determine that the patient is likely to experience onset of relapse of depression when the resulting score is at or above a second threshold value, the second threshold value being lower than the first threshold value, and the processor 302 determines from prior data stored in the memory storage device 310 that prior resulting scores in the preceding predetermined period (e.g., in the most recent two week period) are also above the second threshold value. In this embodiment, the processor 302 may determine that the patient is likely to experience onset of relapse of depression (step 610) when the resulting score is above the second threshold value and the prior data in the memory storage device 310 indicates that the prior resulting score of an immediate prior consecutive iteration of the method 400 is also above the second threshold value. If these criteria are not met, the processor 302 proceeds to step 612. In step 612, result(s) from step 408 and the resulting score(s) obtained using the self-report test(s) as described above are stored in the memory storage device 310 to be used as results generated using prior test data in the next iteration of the method 400.

In an alternative embodiment, if the criteria of steps 602, 606 and 608 are not met, the method 600 may proceed to a further step (not shown) of analyzing the resulting score of step 608. Specifically, a relative change index (CI) is determined as follows:

$$CI = \frac{( {x}_{2} - {x}_{1} )}{{σ}_{1} * \sqrt{2 ( 1 - r )}}$$

where x₂ corresponds to the resulting score based on input from step 602, x₁ corresponds to a baseline score for the patient generated from previous data, σ₁ corresponds to a standard deviation of a set of normative data, and r corresponds to a value representing test reliability of the self-reported test. If the CI is above a predetermined threshold, then the processor 302 may determine that the patient is likely to experience onset of relapse of depression (step 610). Otherwise, the method 600 proceeds to step 612.

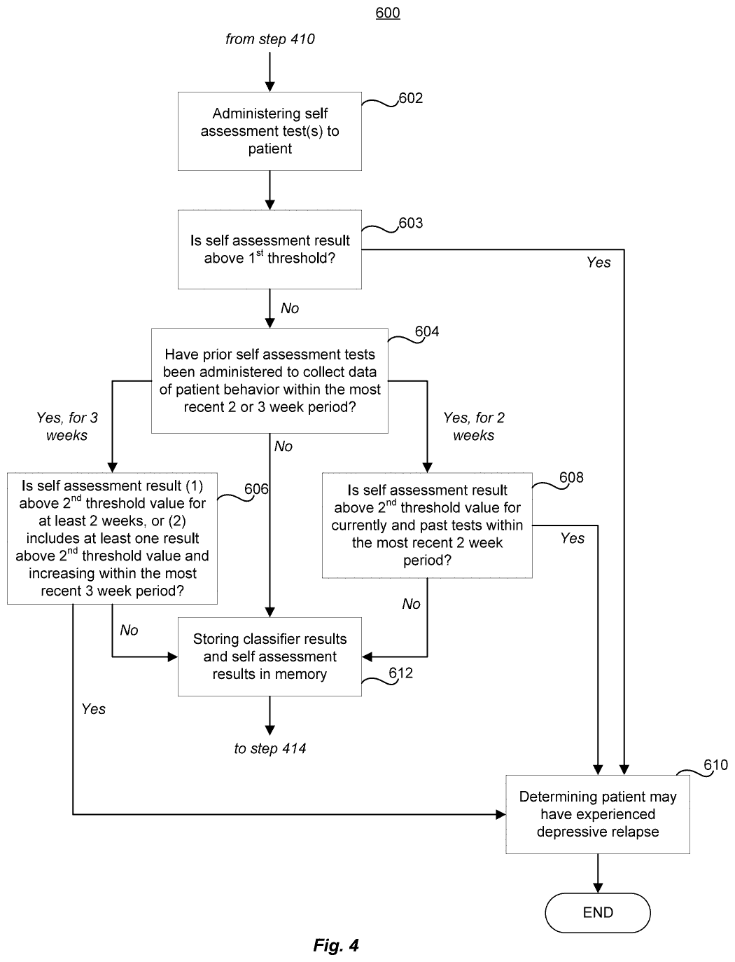

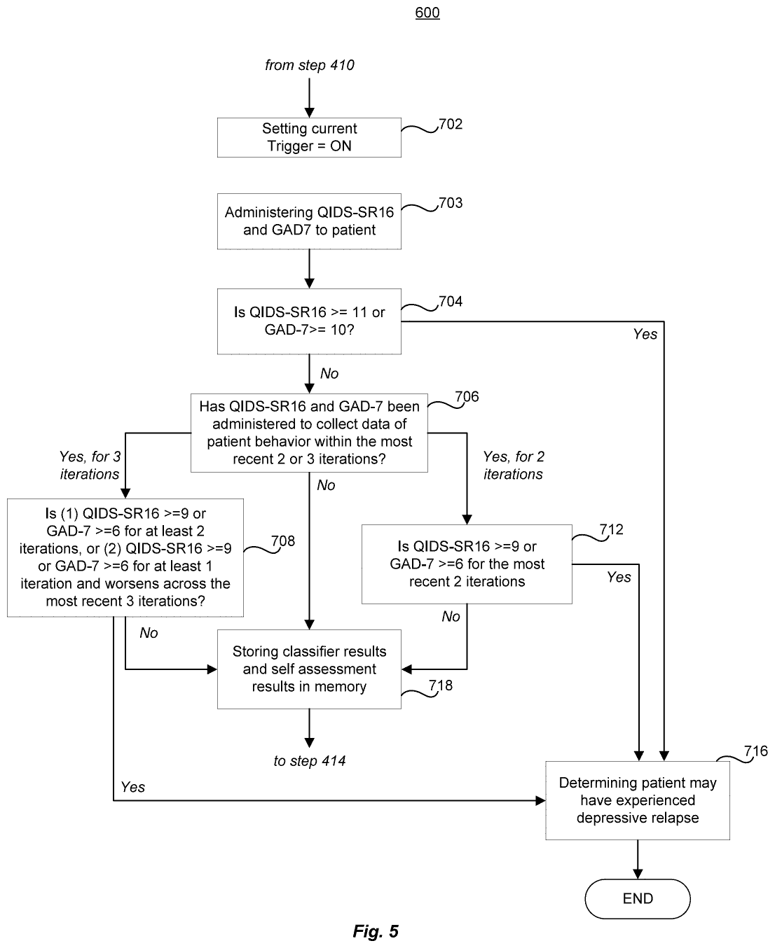

FIG. 5 shows an exemplary embodiment of a method 700 for administering two self-report tests via the computing device 300 and further determining whether the patient is likely to experience onset of relapse of depression. The method 700 is substantially similar to method 600, except as described further below. The method 700 may be used to apply two self-report tests to provide two independent scales for assessing whether the patient is likely to experience onset of relapse of depression, providing further specificity in detecting and/or predicting a patient's relapse into depression. It is noted that the exemplary method 700 shown in FIG. 5 may substitute for method 600 in method 400 for detecting and/or predicting onset of relapse of depression based on actigraphy data of a patient shown in FIG. 2.

In step 702, the processor 302 may set a trigger variable in the memory storage device corresponding to whether the self-report tests are to be administered in the current iteration of the method 400 to a value of “ON.” In step 704, the computing device 300 engages the patient and administers a plurality of survey questions for QIDS-SR16 or GAD-7, in a substantially similar manner as described above in step 602. In one embodiment, step 704 may administer a plurality of survey questions for both QIDS-SR16 and GAD-7. Inputs obtained according to questions for each of QIDS-SR15 and GAD-7 may be separately analyzed by the processor 302 according to steps 705 to 718 as explained further below.

Similar to step 603, in step 705, the processor 302 analyzes data corresponding to the plurality of inputs from the patient in response to the plurality of survey questions for QIDS-SR16 or GAD-7 to determine whether the patient is likely to experience onset of relapse of depression, and if so, proceed to step 716. Specifically, the processor 302 may analyze data corresponding to the plurality of inputs from the patient in response to the survey questions for QIDS-SR16 to generate a QIDS-SR16 score. Similarly, the processor 302 may analyze data corresponding to the plurality of inputs from the patient in response to the survey questions for GAD-7 to generate a GAD-7 score. If the QIDS-SR16 score is greater than or equal to 11, or if the GAD-7 score is greater than or equal to 10, then the method 700 proceeds to step 716.

If the resulting score of the QIDS-SR16 or GAD-7 test do not meet the threshold values described above in step 705, the method 700 proceeds to further analyze the QIDS-SR16 or GAD-7 score with prior data stored in the memory storage device 310. In step 706, the processor 302 determines whether the trigger variables are set to “on” within the most recent two or three consecutive iterations (including the current trigger variable of step 702) of the method 400. In particular, the method 400 is repeated on a weekly basis and the processor 302 analyzes whether prior data stored in the memory storage devices 310 includes an “on” value (e.g., “1” indicating that the trigger is on, and “0” indicating the trigger is off) for the trigger variable for three consecutive iterations (including the current trigger variable of step 702) of the method 400. If so, the method 700 proceeds to step 708. If the processor 302 determines that the trigger variables are set to an “on” value for the most recent two iterations (including the current trigger variable of step 702) of the method 400, then the method 700 proceeds to step 712. Otherwise, the method 700 proceeds to step 716.

Similar to step 606, in step 708, the processor 302 analyzes the QIDS-SR16 scores for the most recent three consecutive iterations of the method 400. If the processor 302 determines from the current QIDS-SR16 score and prior data stored in the memory storage device 310 that the QIDS-SR16 score over the most recent three consecutive iterations of the method 400 has been ≥9 for at least two iterations, then the processor 302 determines that the patient is likely to experience onset of relapse of depression, and proceed to step 716. In addition, if the processor 302 determines from the current QIDS-SR16 score and prior data stored in the memory storage device 310 that at least one QIDS-SR16 score over the most recent three consecutive iterations is ≥9 and that the QIDS-SR16 score has worsened (e.g., shown by a 1 point increase) over the most recent three consecutive iterations, then the processor 302 determines that the patient is likely to experience onset of relapse of depression, and proceed to step 716. Similarly, if the processor 302 determines from the current GAD-7 score and prior data stored in the memory storage device 310 that the GAD-7 score over the most recent three consecutive iterations of the method 400 has been ≥6 for at least two iterations, then the processor 302 determines that the patient is likely to experience onset of relapse of depression, and proceed to step 716. In addition, if the processor 302 determines from the current GAD-7 score and prior data stored in the memory storage device 310 that at least one GAD-7 score over the most recent three consecutive iterations is ≥6 and that the QIDS-SR16 score has worsened (e.g., shown by a 1 point increase) over the most recent three consecutive iterations, then the processor 302 determines that the patient is likely to experience onset of relapse of depression, and proceed to step 716. If neither of these criteria are not met, the processor 302 proceeds to step 718.

Similar to step 608, in step 712, the processor 302 analyzes the QIDS-SR16 scores for the most recent two consecutive iterations of the method 400. If the current QIDS-SR16 score (based on input provided in step 704) is ≥9 and the processor 302 determines from prior data stored in the memory storage device 310 that the prior QIDS-SR16 score in an immediately prior iteration of the method 400 is also ≥9, the processor 302 determines that the patient is likely to experience onset of relapse of depression, and proceed to step 716. Similarly, if the current GAD-7 score (based on input provided in step 704) is ≥6 and the processor 302 determines from prior data stored in the memory storage device 310 that the prior GAD-7 score in an immediately prior iteration of the method 400 is also ≥6, the processor 302 determines that the patient is likely to experience onset of relapse of depression, and proceed to step 716. If these criteria are not met, the processor 302 proceeds to step 718. Similar to step 612, step 718 stores result(s) from step 408 and the QIDS-SR16 and/or GAD7 score(s) as described above in memory storage device 310 to be used as results generated using prior test data in the next iteration of the method 400.

In both steps 610 and 716, the processor 302 determines that the patient is likely to experience onset of relapse of depression. Following such a determination by the processor 302, the processor 302 may direct or output a signal directing an adjustment to a treatment for depression. The treatment for depression may comprise psychotherapy, brain stimulation therapy, or administration of an antidepressant. The antidepressant may be an oral antidepressant, a nasally administered antidepressant, or a transdermal antidepressant. The adjustments to psychotherapy may comprise increasing or decreasing frequency of sessions and/or length of time of each session. The adjustments to brain stimulation therapy may comprise increasing or decreasing frequency and/or intensity of stimulation intervention. The adjustments to administration of an antidepressant may comprise changes to the antidepressant regiment (e.g., increase or decrease dose and/or frequency of antidepressant administration), changes to type or class of antidepressant, or addition of another antidepressant. In some embodiments, the adjustments to antidepressant regimen may be for those patients who are non-treatment resistant (e.g., non-resistant to oral antidepressant therapy). In other embodiments, the adjustments to antidepressant regimen may be for those patients who are continuing to respond to and receive oral antidepressants. In a further embodiment, the adjustments to antidepressant regimen may be for those patients who are treatment resistant to oral antidepressant regiment and the adjustment is addition of another antidepressant administered nasally or transdermally. In another embodiment, following a determination that the patient is likely to experience onset of relapse of depression, the processor 302 may direct or output a signal directing an adjustment to administration of other agents that may be suitable for controlling symptoms of depression, such as, for example, N-methyl-D-aspartate receptor antagonist, ionotropic glutamate receptor antagonist, and esketamine. The adjustments to administration may comprise increasing or decreasing dose and/or frequency of the agent. Alternatively, the adjustment to administration may include substituting the agent in place of an antidepressant, or in addition to an antidepressant.

At the end of each iteration of the method 400, both step 412 and method 600 (if the patient is not identified as having experienced a relapse) proceed to step 414. In step 404, the training actigraphy data is updated to include most recently collected test actigraphy data as part of an updated training actigraphy data used to retrain the at least one anomaly detector in method 500. In one exemplary embodiment, the updated training actigraphy data incorporates the most recently collected test actigraphy data and eliminates training actigraphy data obtained earlier than a most recently predetermined time period. For example, if the training actigraphy data is obtained in a 3-month time period, the most recently collected test actigraphy data is incorporated into the updated training actigraphy data and data older than 3 months from the updated training actigraphy data.

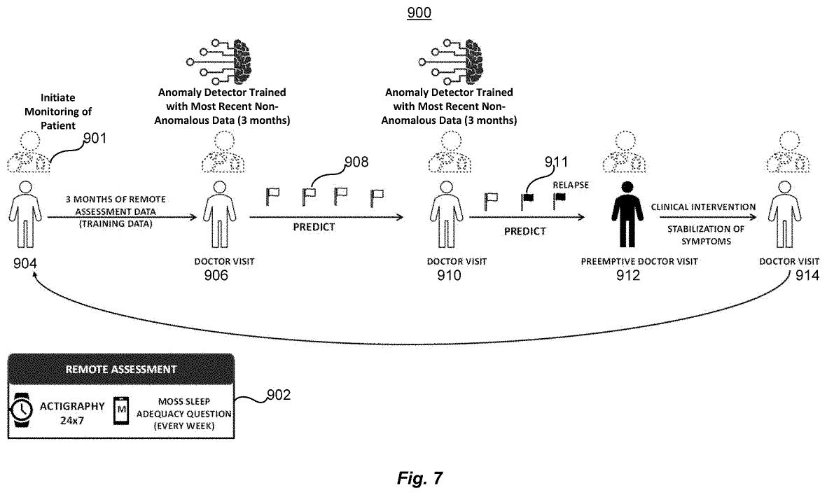

FIG. 7 shows an exemplary timeline 900, as would be experienced by a patient, of the exemplary method 400 of FIG. 2 for detecting and/or predicting relapse of depression of a patient. The patient may initiate the method 400 at time 904 during an initial visit a mental health provider 901. The patient may be provided with an actigraphy device 200 and a computing device 300 for remote assessment 902, in which data is collected from the patient while they are away from the mental health provider 901 during their day to day activities, from time 904 to time 912. In one exemplary embodiment, the actigraphy device 200 may be worn by the patient at all times (e.g., 24 hours a day for 7 days a week) to collect ongoing actigraphy data of the patient from time 904 to time 912. The computing device 300 collects self-report data as inputted by the patient, such as self-report assessment of sleep adequacy. The self-report assessment may be inputted in response to questions relating to the patient's perception of sleep or may include questions from the MOS-S Scale assessment relating to sleep adequacy. In the exemplary timeline 900, the self-report assessment is obtained weekly. Although FIG. 7 shows that remote assessment 902 includes collection of actigraphy data, it is contemplated that remote assessment 902 may also include collection of other types of passive patient data.

Between time 904 and time 906 (e.g., may be a period of 3 month), the actigraphy device 200 and the computing device 300 collects data by remote assessment 902 while the patient has not experienced a relapse (as noted in white in FIG. 7). At time 906, the patient may visit the mental health provider 901 to confirm that the patient has not experienced a relapse between time 904 and time 906. If the patient has not experienced a relapse, data collected between time 904 and time 906 may be used to train an anomaly detector. The anomaly detector is used to screen newly obtained remote assessment data from the patient to determine if the new data is anomalous as compared to previously collected remote assessment data.

Between time 906 and the patient's next visit to the mental health provider 901 at time 910, remote assessment 902 continues to collect data from the patient and iteratively analyze newly collected data using the trained anomaly detector to determine whether the new data is anomalous. For each iteration of the method 400 (an example of which is illustrated in FIG. 7 as flags) the anomaly detector is updated using the most recent three-months of remote assessment data as training data. In the exemplary timeline 900 shown in FIG. 7, each white flag 908 reflects an iteration of the method 400 in which the new remote assessment data is analyzed using the anomaly detector and determined as not anomalous. The period between time 906 and time 910 may be continuously repeated as long as new remote assessment data is not determined to be an anomaly by the anomaly detector and the mental health provider 901 confirms at time 910 that the patient is not experiencing a relapse.

As shown in the exemplary timeline 900, between time 910 and the patient's next schedule visit to the mental health provider 901, remote assessment 902 continues to collect data from the patient and iteratively analyze newly collected data using the trained anomaly detector to determine whether the new data is anomalous. However, the anomaly detector initially determines new remote assessment data as not anomalous (illustrated as white flags 908), but subsequently detects anomalous new data (illustrated as black flags 911) in subsequent iterations. Further data collection is triggered when an anomaly is detected. For example, when an anomaly is detected, the computing device 300 may proceed to method 600 or method 700 to administer at least one self-report test to the patient and further determine whether the patient is likely to experienced onset of a relapse of depression based on the self-report test(s).

In the exemplary timeline 900 shown in FIG. 7, method 600 or method 700 may determine that the patient is likely to have experienced onset of a relapse of depression based on the self-report test(s) and direct the patient to preemptively visit their mental health provider 901 at time 912, prior to their next scheduled visit (not shown) to the mental health provider 901. During the preemptive visit at time 912, the mental health provider 901 may confirm that the patient has experienced a relapse (illustrated in black) and subsequently provide early clinical interventions to the patient to stabilize their depression symptoms. Once the patient is stabilized and symptoms of depression are in remission, as shown at time 914 (illustrated in white), the patient may re-initiate the method 400, returning to time 904 in the exemplary timeline 900.

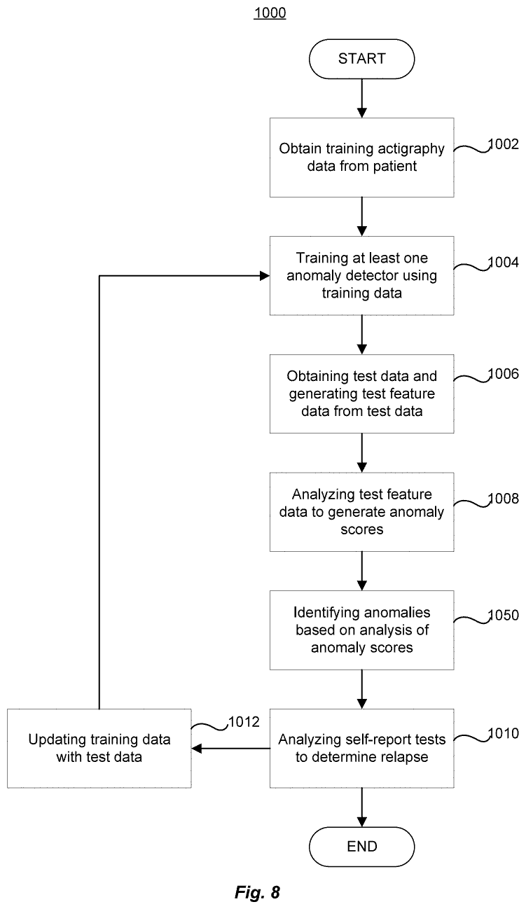

FIG. 8 shows another exemplary method 1000 for detecting and/or predicting relapse of depression of a patient. The exemplary method 1000 is similar to method 400, except as otherwise described below. The exemplary method 1000 utilizes multiple features extracted from actigraphy data and at least one machine-learning anomaly detector that trains the anomaly detector based on the patient's own historic data (n=1, where n is the total number of individuals sampled) to determine likelihood of onset of relapse of depression in a patient. A patient's relapse into depression may be identified using one or more tests as discussed above with respect to method 400. If the computing device 300 determines that the patient has relapsed, the exemplary method 1000 is terminated. The method 1000 may be restarted in a similar manner as method 400 when the patient has returned to remission of depression. Therefore, the method 1000 may be continuously used to train one or more anomaly detectors on all non-relapse actigraphy data recorded from the patient and continuously analyze and detect anomalies in subsequently recorded data. In one example, the anomaly detector(s) is continuously trained on features extracted from actigraphy data obtained during non-relapse time periods through continuous iterations of method 1000. The portion of method 1000 for detecting anomalies in actigraphy data recorded from a patient provides an objective evaluation of the patient to determine if the actigraphy data is anomalous as compared to previously collected actigraphy data from the patient during a time period when the patient is not known to have developed symptom(s) of depression and/or relapse of depression. Once the anomalous instances are identified, a self-report symptomatology (core-symptoms of depression and anxiety) questionnaires algorithm (SRSQA) is used to confirm the relapse signatures. The self-report symptomology questionnaires or SRSQA may be substantially similar to the self-report tests, as described above with respect to the methods 600 or 700 of the method 400.

In step 1002, training data is collected by the device 200 and/or the computing device 300 over a predetermined training time period. The training data comprise actigraphy data collected by the device 200 over the predetermined training time period. The device 200 may be worn by the patient to detect movement of the patient and generate training actigraphy data over the predetermined training time period in a similar manner as described above in step 402. Similar to method 400, the method 1000 also terminates if the computing device 300 determines that the patient has relapsed. Therefore, the training data is collected during non-relapsed time periods.

In step 1004, the training data obtained in step 1002 is used to train at least one anomaly detector for determining a likelihood of onset of relapse of depression, for example, in a manner similar to method 500, except as otherwise noted below. Similar to step 502, the training data in step 1004 is analyzed to extract a plurality of features from the training data to generate training feature data. For example, the training data includes training actigraphy data. Various features are extracted from the training actigraphy data and are included in the training feature data. In particular, the features extracted from the training actigraphy data include monofractal patterns and/or multifractal dynamics of the training actigraphy data, and may further include Sample entropy (SaEn) across different time frames.

For example, the features extracted from the training actigraphy data include fractal patterns determined using the DFA method described above. In this exemplary method 1000, to ensure reliable estimation of F(n) at a time scale n, actigraphy data for the most recent continuous days of activity (at least 2 consecutive days) with no gaps >72 minute (5% of 1440 minutes of activity counts in a day) for each day are used in the DFA method to determine fractal patterns of the actigraphy data. In this exemplary method 1000, the fluctuation exponent (α) for the DFA method is determined at two different time scales as α₁ during 10 (i.e., 10 data points with epoch length of 1 min) minutes to 90 minutes and α₂ during 120 minutes to 600 minutes respectively to capture the distinct regions of activity dynamics. The features extracted from the training actigraphy data also include multifractal dynamics of the actigraphy data using a MFDFA method. In this exemplary method 1300, the multifractal dynamics is determined using the exemplary MFDFA method describe above where the fluctuation of the data is generally represented as:

$${F}_{q} ( s ) = {〈 {\{ {\sum }_{j = 1}^{s} {( {Y}_{v} ( j ) - {p}_{s , v}^{k} ( j ) )}^{2} \}}^{\frac{q}{2}} 〉}^{\frac{1}{q}} .$$

Furthermore, the features extracted from the training actigraphy data include Sample entropy (SaEn) of the training actigraphy data for each day and each of four epochs, morning (6 am to 12 pm), afternoon (12 pm to 6 pm), evening (6 pm to 12 am), and night (12 am to 6 am) determined in the manner described above.

The extracted training feature data may be filtered in the same manner as step 504 and the processor 302 may subsequently modify the filtered training feature data by imputing missing data points to generated imputed training data in a similar manner to step 506. In one example, missing data is imputed a value of 0. The imputed training data may be centered and scaled using population mean and standard deviation (data-mean (population data)/standard deviation (population data) in the same manner as step 508. A power transformation (e.g., a YeoJohnson transformation, or a Box Cox transformation) may also be applied to all features of the imputed training data to generate modified training data having a normalized distribution.

The modified training data is used to train at least one machine-learning anomaly detector for determining a likelihood of onset of relapse of depression. In one example, the anomaly detector models normal behavior based on the training feature data and use prediction error of the model to identify anomalies. Specifically, the anomaly detector is the Enc-Dec AD described above. The encoder of the Enc-Dec AD is trained using the training feature data to learn a vector representation for a time series of the actigraphy data. The modified training data is used to determine the mean (μ) and the standard deviation (Σ) of the Normal distribution [symbol] (μ, Σ) of the time series using Maximum Likelihood Estimation. The μ and Σ are subsequently used by the Enc-Dec AD to determine an anomaly score for assessing whether an anomaly has been detected.

In step 1006, the device 200 obtains test data from the patient similar to step 404 described above. The test data includes test actigraphy data from the patient. The test data may be generated in real-time or may be generated over a desired testing period, such as, for example, a period of w days, e.g., 14 days. In some embodiments, the entirety of the w day period may be generated in step 1006. In other embodiments, the test data may include a portion of previously generated data and new data generated obtained over a desired data collection period (e.g., y number of days) in step 1006. The portion of previously collected data may be for a duration of w-y number of days. For example, the test data may include 13 days of previously generated data and 1 day of new data. The processor 302 analyzes and extracts a plurality of features from the test data in a similar manner as discussed above with respect to step 1004 to generate test feature data.

During step 1006, the computing device 300 may also administer self-report assessment test(s) via the computing device 300 and collect self-report test data from the patients. For example, the self-report tests may be administered on a weekly basis during the desired testing period. Therefore, the self-report tests may not be administered at each iteration of the method 1000. The self-report test(s) may comprise survey questions prompting the patient to input a series of responses along a quantitative scale (e.g., rating on a numerical scale for each symptom) as discussed above with respect to methods 400 and 600. For example, the self-report test(s) may comprise survey questions for prompting the patient to input a series of responses along a quantitative scale for assessing depressive symptoms, mood related cognition, energy/motivation, anhedonia, pain, healthcare utilization, stress/resilience, functioning/disability, health related quality of life, anxiety and/or sleep disturbance. In one embodiment, the self-report test(s) administered during step 1006 comprise evaluations using the VQIDS-SR5 and/or the GAD-7. The VQIDS-SR5 is a patient-reported measure designed to assess the severity of depressive symptoms. The VQIDS-SR5 assesses core depression domains, namely, sad mood, self-outlook, involvement, fatigue, and psychomotor slowing, which are extracted from the QIDS-SR16 to quickly identify a major depressive episode. The total VQIDS-SR5 score is obtained by adding the scores for each of the 5 depression domains. A VQIDS-SR5 score ≥5 correlates to mild depression. A VQIDS-SR5 score ≥6 correlates to moderate to severe depression. The GAD-7 is as described above.

In step 1008, the processor 302 analyzes the test feature data from step 1006 using the anomaly detector(s) trained in step 1002. In particular, the anomaly detector(s) compare the test feature data to the training feature data to generate anomaly scores quantifying the likelihood that the training feature data is an anomaly during the testing period. In one particular embodiment, the anomaly detector is the Enc-Dec AD trained in step 1004. The decoder of the Enc-Dec AD generates a predicted output for the time frame of the test feature data based on the vector representation learned from the training feature data. The Enc-Dec AD determines an anomaly score a⁽ⁱ⁾ as a⁽ⁱ⁾=(e⁽ⁱ⁾−μ)^{T}Σ⁻¹(e⁽ⁱ⁾−μ), where μ and Σ are as determined in step 1004 using the training feature data, and e⁽ⁱ⁾=|x⁽ⁱ⁾−\hat{x}⁽ⁱ⁾|, where x⁽ⁱ⁾ is the test feature data at time point tᵢ and \hat{x}⁽ⁱ⁾ is a reconstructed value at time point tᵢ determined by the Enc-Dec AD. The time point tᵢ is a time point within the testing period in which the training data is collected from the patient. The anomaly score may be calculated across each time point (e.g., for each day) within the testing period in the manner described above.

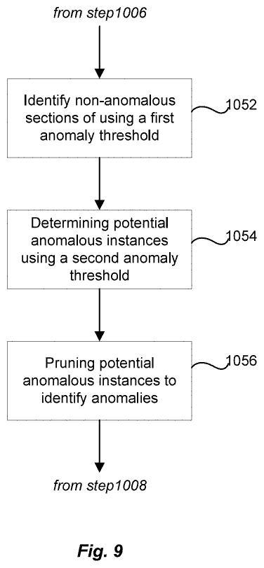

The anomaly scores generated in step 1008 are analyzed may be analyzed using dynamic, data driven thresholds to identify instances of anomalies. FIG. 9 shows an exemplary method 1050 for identifying anomalies using dynamic thresholds. The method 1050 is an unsupervised anomaly scoring method. In one example, given a time series of anomaly scores generated based on test feature data, method 1050 identifies anomalies based on a window of w-days. The w-day window may be represented as a_{w}={a(p+1:p+w)}∀p=0 to (N−w)+1 in steps of 1-day. N is the total number of days actigraphy data has been recorded from the patient. In some examples, N may be the total number of days of the training data and the testing data collected from the patient. The window may extend across any suitable number of days, for example, w may be from 1 to 30 days. In particular, w is the same value as the desired testing period, discussed above in step 1006. In a preferred embodiment, w=14 days. For each iteration across the window of w-day, in order to determine the data driven thresholds for anomaly detection, historical anomaly scores, namely, aₐₗₗ={a(1:T_{c})}∀T_{c}=w to N in steps of 1-day (which includes a_{w}) is used. The historical anomaly scores (aₐₗₗ) may comprise, or consist of, anomaly scores generated based on training feature data and test feature data.

In step 1052, non-anomalous sections of aₐₗₗ are identified as aₐₗₗ_{ₙₒₙ₋ₐₙₒₘₐₗₒᵤₛ}. Specifically, the historical anomaly scores (aₐₗₗ) is used to determine a first anomaly threshold, ∈ₐₗₗ, above which an anomaly score in aₐₗₗ may indicate a potential anomaly. Sections of aₐₗₗ having anomaly scores less than ∈ₐₗₗ are identified as non-anomalous and sections of aₐₗₗ having anomaly scores greater than ∈ₐₗₗ are identified as anomalous. The first anomaly threshold, ∈ₐₗₗ, is determined as:

∈ₐₗₗ=μ(aₐₗₗ)+zₐₗₗσ(aₐₗₗ),

where μ(aₐₗₗ) is the mean of the anomaly scores of aₐₗₗ, σ(aₐₗₗ) is the standard deviation of the anomaly scores of aₐₗₗ, and zₐₗₗ is a value between 1 and 10 indicating the number of standard deviations at which ∈ₐₗₗ is set above μ(aₐₗₗ). In one example, zₐₗₗ is set to two indicating that the number of standard deviations ∈ₐₗₗ is set above μ(aₐₗₗ). The anomalous instances and the corresponding sequence of anomalous instances (each anomalous sequence being 1 time point before and 1 time point after each anomalous instance) in aₐₗₗ above ∈ₐₗₗ are removed to determine as aₐₗₗ_{ₙₒₙ₋ₐₙₒₘₐₗₒᵤₛ}, which are non-anomalous sections of aₐₗₗ.

In step 1054, potential anomalous instances in a_{w} are identified. A second anomaly threshold, ∈ₐₗₗ_{ₙₒₙ₋ₐₙₒₘₐₗₒᵤₛ}, above which an anomaly score in a_{w} may indicate a potential anomaly, is determined based on aₐₗₗ_{ₙₒₙ}₋ₐₙₒₘₐₗₒᵤₛ. In particular, the second anomaly threshold, ∈ₐₗₗ_{ₙₒₙ₋ₐₙₒₘₐₗₒᵤₛ}, is determined as:

∈ₐₗₗ_{ₙₒₙ₋ₐₙₒₘₐₗₒᵤₛ}=μ(aₐₗₗ_{ₙₒₙ}₋ₐₙₒₘₐₗₒᵤₛ)+zₐₗₗ_{ₙₒₙ}₋ₐₙₒₘₐₗₒᵤₛσ(aₐₗₗ_{ₙₒₙ}₋ₐₙₒₘₐₗₒᵤₛ),

where μ(aₐₗₗ_{ₙₒₙ}₋ₐₙₒₘₐₗₒᵤₛ) is the mean of the anomaly scores of aₐₗₗ_{ₙₒₙ}₋ₐₙₒₘₐₗₒᵤₛ, σ(aₐₗₗ_{ₙₒₙ}₋ₐₙₒₘₐₗₒᵤₛ) is the standard deviation of the anomaly scores of aₐₗₗ_{ₙₒₙ}₋ₐₙₒₘₐₗₒᵤₛ, and zₐₗₗ_{ₙₒₙ}₋ₐₙₒₘₐₗₒᵤₛ is a value between 1 and 10 indicating the number of standard deviations at which ∈ₐₗₗ_{ₙₒₙ₋ₐₙₒₘₐₗₒᵤₛ} is set above μ(aₐₗₗ_{ₙₒₙ}₋ₐₙₒₘₐₗₒᵤₛ). In one example, zₐₗₗ_{ₙₒₙ}₋ₐₙₒₘₐₗₒᵤₛ is set to two indicating that the number of standard deviations ∈ₐₗₗ_{ₙₒₙ}₋ₐₙₒₘₐₗₒᵤₛ is set above μ(aₐₗₗ_{ₙₒₙ}₋ₐₙₒₘₐₗₒᵤₛ). The non-anomalous sections of aₐₗₗ (aₐₗₗ_{ₙₒₙ}₋ₐₙₒₘₐₗₒᵤₛ) is used to determine the second anomaly threshold, ∈ₐₗₗ_{ₙₒₙ}₋ₐₙₒₘₐₗₒᵤₛ to mitigate scenarios of large outlier peaks present in aₐₗₗ that may cause missed detection of anomalous instances in a_{w}. If use of the second anomaly threshold, ∈ₐₗₗ_{ₙₒₙ}₋ₐₙₒₘₐₗₒᵤₛ, does not identify any anomalous instances in a_{w}, then local maxima observed in a_{w} (local max (a_{w})) such that local max (a_{w})≥max(aₐₗₗ_{ₙₒₙ₋ₐₙₒₘₐₗₒᵤₛ}) may be included as potential anomalous instances in a_{w}.

In step 1056, the potential anomalous instances identified in step 1054 are pruned to identify those instances that are most likely to be an anomaly. First, the potential anomalous instances in a_{w} identified in step 1054 are removed from to a_{w} to obtain a_{wnon-anomalous} which are non-anomalous sections of a_{w}. For each anomaly sequence (a_{w}_{_{anomalous_seq}}), each anomalous sequence being 1 time point before and 1 time point after each anomalous instance, a percent decrease d⁽ⁱ⁾ at time point tᵢ is determined as:

$$d ( i ) = \frac{{a}_{{a}_{{w}_{anomalous\_seq}}}^{( i )} - max ( {a}_{{w}_{n o n - a n o m a l o u s}} )}{{a}_{{a}_{{w}_{anomalous\_seq}}}^{( i )}}$$

where i is an identifier corresponding to each anomalous instance. Anomalous sequences having a d⁽ⁱ⁾≥0.3 are retained in step 1056 and identified as anomalies. If no anomalous sequences are retained and if there are ≥2 anomalous peaks with d<0.3, the anomalous sequence corresponding to the maximum percent decrease is retained as a possible anomalous instance.

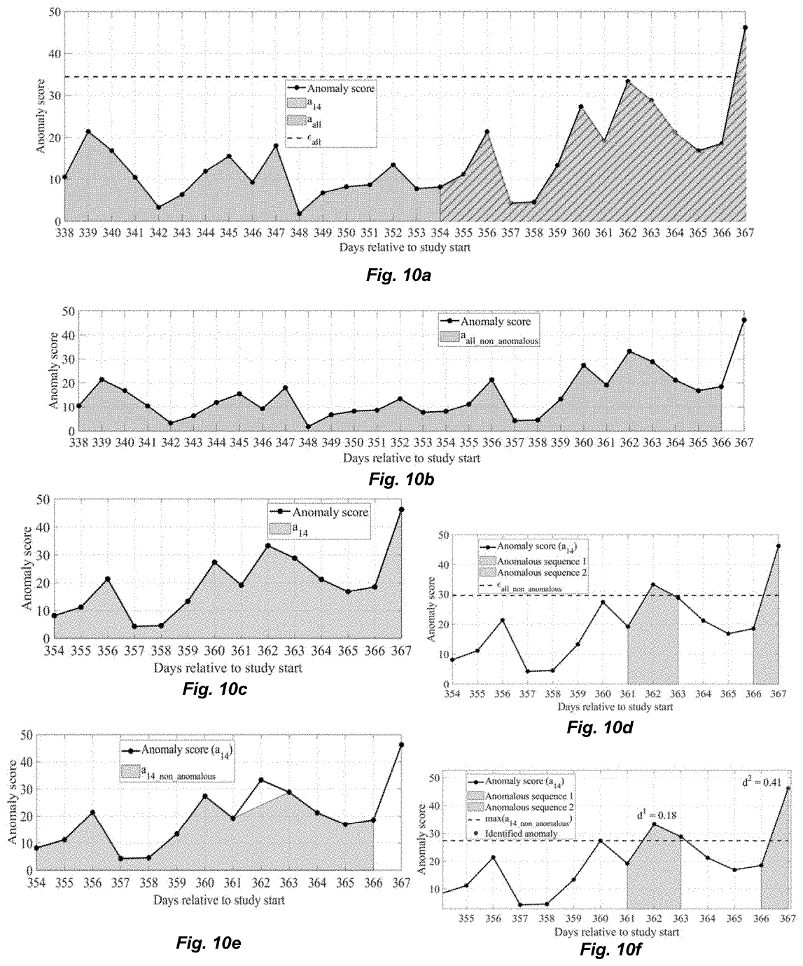

FIGS. 10a-f show an example of time series of anomaly scores for actigraphy data collected during an exemplary period from a patient and analyzing a most recently acquired 14 days of actigraphy data to identify anomalous instances according to steps 1052 through 1056. FIG. 10a shows an exemplary timeline for actigraphy data collected from day 338 after initiation a study to day 367. In this exemplary embodiment, the anomaly scores from day 338 to day 354 are generated from training feature data based on actigraphy data collected during a period from day 338 up to but not including day 354. The anomaly scores starting from day 354 to day 367 are generated from test feature data based on actigraphy data collected during a period from day 354 to day 367. The shaded areas shown in FIG. 10a correspond to aₐₗₗ for this exemplary embodiment. The a_{w} for this exemplary embodiment is a period of 14 days. Therefore, a₁₄ will be used herein for this series of anomaly scores in this example. As can be seen in FIG. 10a, a₁₄ extends through a period from day 354 to day 367 (shown with cross hatches) and overlaps completely with aₐₗₗ. The ∈ₐₗₗ for this exemplary time series is shown as a horizontal dotted line across FIG. 10a. As can be seen, the anomaly score for day 367 is greater than ∈ₐₗₗ. Therefore, the anomaly score for day 367 is excluded from aₐₗₗ_{ₙₒₙ}₋ₐₙₒₘₐₗₒᵤₛ of this example, which is shown as the shaded area in FIG. 10b. FIG. 10c shows the same a₁₄ time series of anomaly scores from FIG. 10a as the shaded area. FIG. 10d shows potential anomalous instances in a₁₄ that are identified according to step 1054. The potential anomalous instances and corresponding anomalous sequences (1 day before and 1 day after each anomalous instance) are shown as the shaded area in FIG. 10d. The ∈ₐₗₗ_{ₙₒₙ₋ₐₙₒₘₐₗₒᵤₛ} for this example is shown as a horizontal dotted line across FIG. 10d. As can be seen in FIG. 10d, the anomaly score for day 362 and day 367 greater than ∈ₐₗₗ_{ₙₒₙ₋ₐₙₒₘₐₗₒᵤₛ}. Therefore, the anomaly scores for day 362 and day 367 are excluded from a_{w}_{ₙₒₙ₋ₐₙₒₘₐₗₒᵤₛ}, which is shown as the shaded are in FIG. 10e. The potential anomalous instances shown in FIG. 10d are further pruned according to step 1054. FIG. 10f shows a percentage decrease d⁽ⁱ⁾ for each of the two anomalous instances identified in FIG. 10d. The percentage decrease d¹ for the instance at day 362 is below 0.3 and is therefore pruned during step 1056. The percentage decrease d² for the instance at day 367 is above 0.3 and is therefore retained and identified as an anomaly.

In step 1010, the computing device 30 analyzes self-report test data corresponding to and/or within a desired period from any anomaly identified by method 1050 to determine whether the anomaly indicates that the patient is likely to experience onset of relapse of depression. For each anomaly, corresponding self-report test data or self-report data within a desired period from the anomaly are analyzed to determine whether or not the anomaly indicates that the patient is likely to experience onset of relapse of depression. In particular, step 1008 determines whether or not an anomaly is identified by method 1050 within the first w days or w/2 days of test feature data.

In one example, w is 14 days and the anomalies further analyzed in step 1008 are identified based on the presence of anomalous instance within the most recent 7 days of the 14 day period. The method 1000 considers whether or not to collect the self-report test data in step 1006 of this example on a weekly (i.e., every 7 days) basis. For each week where an anomaly has been identified by method 1050, self-report test data for the week identified are collected in step 1006. In addition, for each week where an anomaly was identified in a previous week by the method 1000 (i.e., a week after the identified anomalous week) self-report test data are also collected in step 1006. Self-report test data from both of these weeks are analyzed to determine whether the anomaly indicates that the patient is likely to experience onset of relapse of depression. Therefore, self-report test data for a week closest to the identified anomaly and a following week the identified anomalous week are analyzed to determine whether the anomaly indicates that the patient is likely to experience onset of relapse of depression. The self-report test data comprise responses to evaluations using the VQIDS-SR5 and/or the GAD-7. Specifically, the self-report test data comprise responses to evaluations using the VQIDS-SR5 and the GAD-7, the combination of which utilizes 12 questions in total. The self-report test is administered to the patient when an anomaly has been detected in the week identified and may also be administered to the patient for the week after the identified anomalous week. In one example, the self-report test is administered to the patient when anomaly has been detected in the week identified and if the self-report data comprises evaluations (e.g., VQIDS-SR5 and/or GAD-7 scores) above a set of high predetermined threshold(s) (e.g., VQIDS-SR5≥6 and/or GAD-7≥10), then the method 1000 determines that the patient is likely to experience onset of relapse of depression. However, if the self-report test data comprises evaluations above a set of lower predetermined threshold(s) (e.g., VQIDS-SR5≥5 and/or GAD-7≥5), then the method 1000 continues to be iterated and obtains further self-report test data a week after the identified anomalous week. If the self-report test data includes evaluations that are above the set of lower predetermined threshold(s) (e.g., VQIDS-SR5≥5 and/or GAD-7≥5) for two consecutive weeks, then the method 1000 determines that the patient is likely to experience onset of relapse of depression. The method 1000 does not require administration of the self-report test during other weeks and can therefore reduce burden to patients who are being monitored by repeat iterations of the method 1000. Furthermore, the method 1000 in this example identifies anomalous instances and indicates that the patient is likely to experience onset of relapse of depression after confirmation using self-report test data (specifically, 2 weeks of self-report test data) and therefore, reduces instances of false positives as compared to detection of anomalies in the test data (including test actigraphy data) alone or identification of relapse based on weekly collection of self-report test data alone.

The patient may be determined as likely to experience onset of relapse of depression when the VQIDS-SR5 score is ≥6 for either the week where an anomaly has been identified or the week after to the identified anomalous week. Alternatively, the patient may be determined as likely to experience onset of relapse of depression when the GAD-7 score is ≥10 either for the week where an anomaly has been identified or the week after to the identified anomalous week. In another example, the patient may be determined as likely to experience onset of relapse of depression when the VQIDS-SR5 score is ≥5 and/or the GAD-7 score is ≥5 for the week where an anomaly has been identified and for the week after to the identified anomalous week. Following a determination by the processor 302 that the patient is likely to experience onset of relapse of depression, the processor 302 may direct or output a signal directing an adjustment to a treatment for depression. The treatment for depression may comprise psychotherapy, brain stimulation therapy, or administration of an antidepressant as discussed above.

In step 1012, which is at the end of each iteration of the method 1000, the training actigraphy data is updated to include most recently collected test actigraphy data as part of an updated training actigraphy data used to retrain the at least one anomaly detector in step 1004. In one exemplary embodiment, the updated training actigraphy data incorporate the most recently collected test actigraphy data and eliminates training actigraphy data obtained earlier than a most recently predetermined time period. Similar to method 400, the exemplary method 1000 may be repeated continuously or may be iterated at desired frequencies, e.g., daily, weekly, bi-weekly, etc. In one particular embodiment, the exemplary method 1000 is iterated daily. In one example, the exemplary method 1000 is repeated is iterated at a frequency of every y number of days, where y is less than or equal to the length of w days in method 1050. In particular, y is the same value as the desired data collection period, discussed above in step 1006. In one example y is 1 day and w is 14 days.

Although the method 1000 is described above for detecting and/or predicting relapse of depression of a patient. It is contemplated that method 1000 can be modified for detecting and/or predicting relapse of other neurological disorders, in particular, those neurological disorders where changes in activity patterns (as recorded by actigraphy data) indicate relapse of such neurological disorders, such as schizophrenia and bipolar disease. In particular, the self-report test data collected during step 1006 and analyzed in step 1010 may be modified. The self-report test data may be collected from self-report test(s) for quantitatively assessing symptoms other neurological disorders. For example, a self-report test for schizophrenia may be the Symptoms of Schizophrenia (SOS) Inventory. As another example, a self-report test for bipolar diseases may be the Hypomanic Personality Scale, the Mood Disorder Questionnaire, the Temperament Evaluation of the Memphis, Pisa, Paris, and San Diego-Autoquestionnaire version, the Bipolar Spectrum Diagnostic Scale, the General Behavior Inventory, and the Hypomania Checklist.

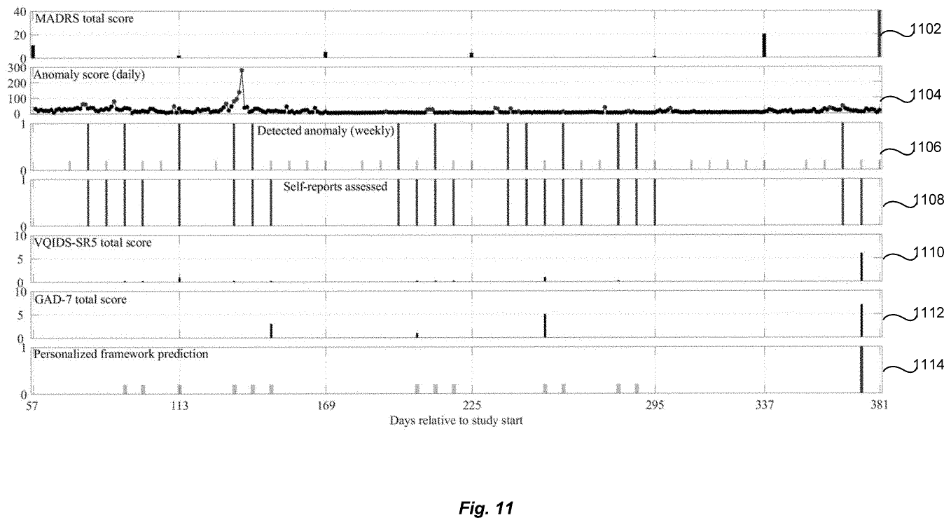

FIG. 11 shows an example of an implementation of the method 1000 across a sample time series of data collected from day 57 to day 381. In particular, FIG. 11 includes a sample time series of MADRS scores 1102 along with corresponding anomaly scores 1104 generated from actigraphy collected from a patient analyzed according to the Enc-Dec AD described in step 1008, and corresponding VQIDS-SR5 scores 1110 and GAD-7 scores 1112 based on self-report collected from the patient. The time series shown as 1106 represents weekly assessments of the anomaly scores 1104 where a short bar indicates that the anomaly scores of that week do not include any anomalies according to method 1050 and a tall bar indicates that the anomaly scores of that week include potentially anomalous instances as identified by method 1050. The time series shown as 1108 indicates those weeks when self-report test data are analyzed according to step 1008 as tall bars. The time series shown as 1114 illustrates a time series of determinations of whether the patient is likely to experience onset of relapse of depression. A tall bar indicates that the patient is likely to experience onset of relapse of depression. A short bar indicates that the self-report test data were analyzed and as can be seen in time series 1114, step 1008 identifies a likely relapse of depression before day 381 where a MADRS score of the patient rises to above 22 indicating a relapse of depression.

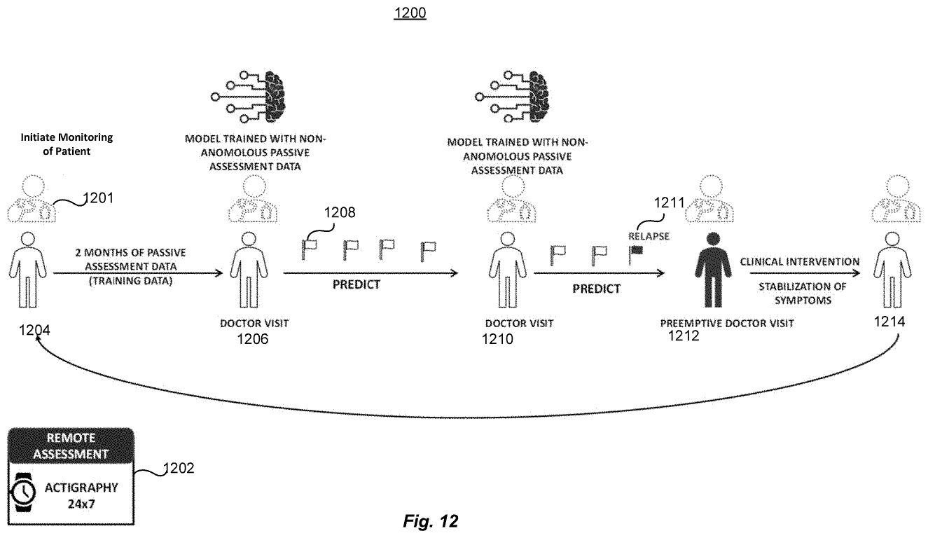

FIG. 12 shows an exemplary timeline 1200, as would be experienced by a patient of the exemplary method 1000 of FIG. 10 for detecting and/or predicting relapse of depression of a patient. The patient may initiate the method 1000 at time 1204 during an initial visit to a mental health provider 1201 and is provided with an actigraphy device 200, in which actigraphy data is collected from the patient during their day to day activities between visits to the mental health provider 1201, from time 1204 to time 1212. In one exemplary embodiment, the actigraphy device 200 may be worn by the patient at all times (e.g., 24 hours a day for 7 days a week) to collect ongoing actigraphy data of the patient from time 1204 to time 1212. Although FIG. 12 shows that remote assessment 1202 includes collection of actigraphy data, it is contemplated that remote assessment 1202 may also include collection of other types of passive patient data. Between time 1204 and time 1206 (e.g., may be a period of 2 months), the device 200 collects data by remote assessment 1202 while the patient has not experienced a relapse as training data (as noted in white in FIG. 12). At time 1206, the patient may visit the mental health provider 1201 to confirm that the patient has not experienced a relapse between time 1204 and time 1206. If the patient has not experienced a relapse, data collected between time 1204 and time 1206 may be used to train an anomaly detector (e.g., the Enc-Dec AD described above). The anomaly detector is used to analyze newly obtained remote assessment data from the patient to determine if the new data indicates that the patient is likely to experience onset of relapse of depression.

Between time 1206 and the patient's next visit to the mental health provider 1201 at time 1210, remote assessment 1202 continues to collect data from the patient. The remote assessment 1202 also includes the computing device 300 collecting self-report data inputted by the patient, as described above in step 1006. In the exemplary timeline 1200, the self-report assessment is obtained weekly. The computing device 300 iteratively analyzes newly collected actigraphy and self-report data according to method 1000 determine whether the new data indicates that the patient is likely to experience onset of relapse of depression. For each iteration of the method 1000 (an example of which is illustrated in FIG. 12 as flags), the anomaly detector is updated using data including the most recent remote assessment data as training data. In the exemplary timeline shown in FIG. 12, each white flag 1208 reflects an iteration of the method 1000 in which the new remote assessment data is analyzed using the method 1000 and determined as not indicating that the patient is likely to experience onset of relapse of depression. The period between time 1206 and time 1210 may be continuously repeated as long as new remote assessment data is not determined to indicate that the patient is likely to experience onset of relapse of depression, and the mental health provider 1201 confirms at time 1210 that the patient is not experiencing a relapse.

As shown in the exemplary timeline 1200, between time 1210 and the patient's next schedule visit to the mental health provider 1201, remote assessment 1202 continues to collect data from the patient and iteratively analyze newly collected data using the method 1000 to determine whether the patient is likely to experience onset of relapse of depression. However, as illustrated in the example of FIG. 12, the anomaly detector determines new remote assessment data as indicating that the patient is not likely to experience onset of relapse of depression (illustrated as white flags 1208), but subsequently detects that the patient is likely to experience onset of relapse of depression (illustrated as black flags 1211) in subsequent iterations.

When the patient is determined as likely to have experienced onset of a relapse of depression, the computing device 300 instructs the patient to preemptively visit their mental health provider 1201 at time 1212, prior to their next scheduled visit (not shown) to the mental health provider 1201. During the preemptive visit at time 1212, the mental health provider 1201 may confirm that the patient has experienced a relapse (illustrated in black) and subsequently provide early clinical interventions to the patient to stabilize their depression symptoms. Once the patient is stabilized and symptoms of depression are in remission, as shown at time 1214 (illustrated in white), the patient may re-initiate the method 1000, returning to time 1204 in the exemplary timeline 1200.

A patient at risk of relapse may be continuously monitored according to method 1000 described above. Actigraphy data is continuously collected and patient's relapse status is determined by a mental health provider during regularly scheduled (e.g., bimonthly) visits. At each scheduled visit, if the mental health provider determines that the patient has not relapsed, then all of the actigraphy data collected until the visit are used to train an anomaly detector, specifically, the Enc-Dec AD. The trained model is used to detect anomalous instances in the subsequent clinical visit activity data. These anomalous instances are pruned and the remaining instances are then confirmed by self-reported symptomatology assessments for relapse risk. This process continues with every non-relapse visit to the mental health provider and the Enc-Dec AD is retrained. The retrained Enc-Dec AD is used to identify anomalies in subsequently acquired actigraphy data. Once the method 100 determines that the patient is likely to experience onset of relapse of depression, the computing device 300 may instruct the patient to contact their mental health provider. Alternatively, the computing device 300 may transmit an alert to the patient's mental health provider. The mental health provider may follow up with the patient upon receipt of the alert, such as, for example, a preemptive appointment or a phone call from the mental health provider's office to check on the patient's symptoms. Based on the mental health provider's judgment, an early intervention if needed may be taken which could eventually prevent an impending relapse and lead to better patient outcomes.

Those skilled in the art will understand that the exemplary embodiments described herein may be implemented in any number of manners, including as a separate software module, as a combination of hardware and software, etc. For example, the exemplary methods may be embodiment in one or more programs stored in a non-transitory storage medium and containing lines of code that, when compiled, may be executed by one or more processor cores or a separate processor. A system according to one embodiment comprises a plurality of processor cores and a set of instructions executing on the plurality of processor cores to perform the exemplary methods discussed above. The processor cores or separate processor may be incorporated in or may communicate with any suitable electronic device, for example, on board processing arrangements within the device or processing arrangements external to the device, e.g., a mobile computing device, a smart phone, a computing tablet, a computing device, etc., that may be in communications with at least a portion of the device.

### EXAMPLE

### Example I

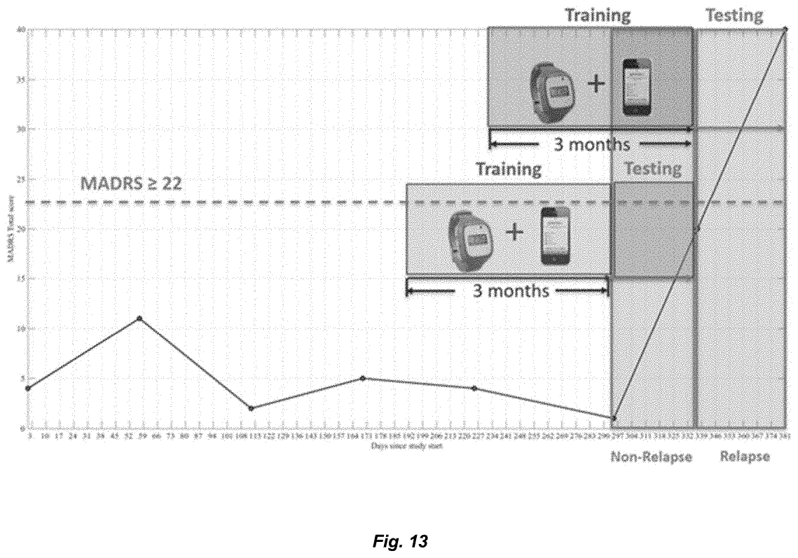

In Example I, an exemplary actigraphy device 200 is provided to a patient to collect actigraphy data and determine relapse of depression in the patient according to the exemplary methods 400 and 700 described above. In addition, the patient is asked to provide daily a quantitative assessment of perception of sleep in response to two self-report assessment questions: (1) do you feel rested; and (2) do you feel you have gotten enough sleep. The inputs from these self-report assessment questions are used to generate training self-report data, which is included in the training data used to train the anomaly detectors used in Example I. The patient in Example I met the DSM-5 diagnostic criteria for nonpsychotic, recurrent MDD within a previous 24-month period and was taking an oral antidepressant, but did not meet the criteria for a major depressive episode at initiation of actigraphy monitoring with the actigraphy device 200. As shown in FIG. 13, training actigraphy data was collected for a period of 3 months and updated as the method 400 iterated every week. The MADRS test was administered to the patient approximately every 8 weeks by a mental health provider in addition to collecting patient actigraphy data daily and iterating method 400 every week to provide a separate test from patient actigraphy for identifying whether the patient has experienced a relapse of depression. This separate test determines the patient to have relapsed based on an initial MADRS score ≥22 followed by a verification visit to a mental health provider where the mental health provider determines that the patient's CGI-S score increased by 2 from a baseline obtained prior to initiating method 400 (i.e., at or prior to day 1) or changes type of medication or dosage of medication within 14 days from when the patient initially experienced a MADRS total score ≥22. Under this test, the patient is also considered to have relapsed regardless of the MADRS score if the patient is hospitalized for worsening of depression, has suicidal ideation with intent or suicidal behavior, or is otherwise indicated by a mental health provider as having relapsed. This separate test also determines a patient to be non-relapsed after an initial MADRS score ≥22 followed by a verification visit to a mental health provider where MADRS score decreased to below 22 and CGI-S score showed no change greater than 2 from the baseline and medication for the patient was also not changed within 14 days from when the patient initially experienced a MADRS total score ≥22. As show in FIG. 13 and listed below in Table 2, the patient in Example I was determined by this separate test to be non-relapsed from day 297 to day 332 and to be relapsed from day 339 to day 381. It is noted that because the MADRS tests is administered in 8 week intervals, relapse of the patient is detected using the MADRS scale on day 381, but the patient is considered to have possibly been in relapse starting from immediately after the previous administration of the MADRS test on day 339 to day 381 shown in FIG. 13 and Table 3.

Example I utilizes a processor 302 of a computing device 300 to analyze the patient's actigraphy data according to the exemplary method 400 for detecting onset of relapse of depression based on actigraphy data of a patient, described above, and iterated on a weekly basis. Example I utilizes two separate anomaly detectors: a one class SVM anomaly detector and an IF anomaly detector to determine a likelihood of relapsed using the actigraphy data. As shown in Table 2, the processor 302 using the one SVM anomaly detector reports a value of 0 when it determines that the test data is consistent with the training data (e.g., not likely to correspond to a relapse) and a value of 1 when it determines that the test data is an anomaly (e.g., likely to correspond to a relapse). The computing device 300 using the IF anomaly detector reports an anomaly score corresponding to a likelihood that the test data corresponds to an anomaly. Furthermore, Example I utilizes method 700 to administer two self-report tests via the computing device 300 and further determine whether the patient is likely to have experienced onset of relapse of depression. Specifically, Example I administers the QIDS-SR16 and/or GAD-7 tests and analyzes the current and prior results from the most recent three weeks to determine whether the patient is at risk of a relapse. In this example, data from the most recent three weeks may be analyzed as shown below in Table 2.

TABLE 2

| Most Recent 3- | |
|---|---|
| Weeks of Data | Determine as at Risk of Relapse When |
| 1 week of test | Current QIDS-SR16 score ≥11 or Current |
| data available | GAD-7 score ≥10 |
| 2 weeks of test | Current QIDS-SR16 score ≥11 or Current GAD-7 |
| data available | score >=10 or QIDS-SR16 score ≥9 (for 2 weeks) |
| | or GAD-7 score ≥6 (for 2 weeks) |
| 3 weeks of test | Current QIDS-SR16 ≥11 score or Current GAD-7 |
| data available | score ≥10 or QIDS-SR16 score ≥9 (for at least 2 |
| | weeks) or GAD-7 score ≥6 (for at least 2 weeks) or |
| | QIDS-SR16 score ≥9 (at least 1 time) with symptom |
| | worsening (e.g., 1 point increase in QIDS-SR16 |
| | score over the 3 week period) or GAD-7 score ≥6 |
| | (at least 1 time) with symptom worsening (e.g., 1 |
| | point increase in GAD-7 score over the 3 week |
| | period) |

It is noted that in Table 2, the number of weeks of test data available refers to the number of most recent consecutive weeks that QIDS-SR16 and/or GAD-7 scores are available within the most recent three-weeks of data. The QIDS-SR16 and GAD-7 scores for Example I are reported below in Table 3.

TABLE 3

| | | QIDS- | | SVM | IF | |
|---|---|---|---|---|---|---|
| | MADRS | SR16 | GAD-7 | Anomaly | Anomaly | |
| Day | Determination | Score | Score | Detector | Detector | Trigger |
| 297 | Non-Relapsed | 2 | 0 | 1 | 0.55 | NA |
| 304 | Non-Relapsed | 0 | 0 | 0 | 0.53 | NA |
| 311 | Non-Relapsed | NA | NA | 1 | 0.52 | NA |
| 318 | Non-Relapsed | NA | NA | 1 | 0.55 | 1 |
| 325 | Non-Relapsed | 2 | 1 | 1 | 0.56 | 1 |
| 332 | Non-Relapsed | 7 | 1 | 0 | 0.53 | NA |
| 339 | Relapsed | 5 | 1 | 0 | 0.50 | NA |
| 346 | Relapsed | 7 | 2 | 0 | 0.50 | NA |
| 353 | Relapsed | 9 | 5 | 1 | 0.49 | NA |
| 360 | Relapsed | 8 | 7 | 1 | 0.55 | 1 |
| 367 | Relapsed | NA | NA | 1 | 0.50 | 1 |
| 374 | Relapsed | 11 | 7 | 1 | 0.50 | 1 |
| 381 | Relapsed | 13 | 9 | 1 | 0.51 | NA |

As shown in Table 3 above, Example I showed that actigraphy data collected from a patient and analyzed by a computing device would identify that a patient is at risk of relapse by day 381 based on patient actigraphy data and scores obtained using QIDS-SR16 and GAD-7 tests. This identification is within 7 days from when the MADRS analysis as described above would indicate that the patient has experienced a relapse of depression. This data demonstrates that use of patient actigraphy data in Example I identified relapse of depression within 7 days from its occurrence and thereby providing an early identification of relapse and enabling an earlier ability to direct changes to the patient's treatment in response to relapse.

### Example II

In Example II, exemplary actigraphy devices 200 were provided to 41 patients who subsequently relapsed. Data for Example II were collected in a similar manner as described above for Example I. The actigraphy devices 200 collected actigraphy data and the computing device 300 collected the patient's quantitative assessment of perception of sleep in response to the two self-report assessment questions described above in Example I. The data was analyzed by the processor 302 of the computing device 300 to detect and/or predict onset of relapse of depression in the patient according to the same method as describe above in Example I using an SVM anomaly detector. Performance metrics for Example II is shown below in Table 4.

TABLE 4

| Performance Metrics | Values |
|---|---|
| True Positive | 30 |
| True Negative | 25 |
| False Positive | 16 |
| False Negative | 10 |
| Accuracy | 0.68 |
| Sensitivity | 0.75 |
| Specificity | 0.61 |
| Balanced Accuracy (BAC) | 0.68 |
| Positive Predictive Value (PPV) | 0.65 |
| Negative Predictive Value (NPV) | 0.71 |
| DOR | 4.69 |
| F₁-measure | 0.70 |

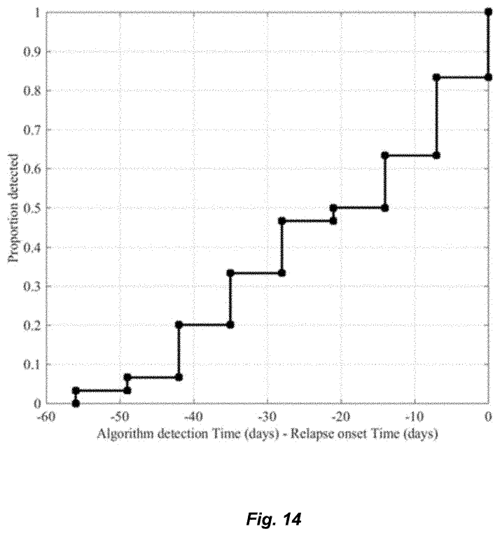

FIG. 14 shows data corresponding to proportion of the group of 30 true positive patients determined as likely to have relapsed over amount of time prior to actual onset of relapse as determined by the MADRS method described in Example I. The data of FIG. 14 shows that 83% of the 30 true positive patients were identified as likely to have relapsed using actigraphy data 7 days or more earlier than actual relapse onset.

### Example III

In Example III, exemplary actigraphy devices 200 were provided to approximately 330 subjects where about 88 subjects relapsed. The actigraphy devices 300 collected actigraphy data and training self-report data, as described above in Example I, and the data was analyzed by the computing device 400 to determine relapse of depression in the patient according to the same method as describe above in Example I.

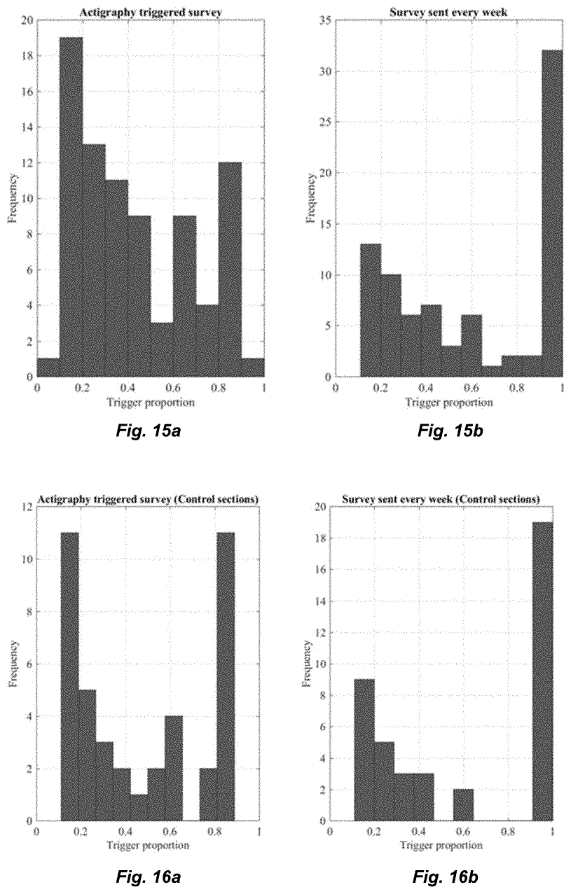

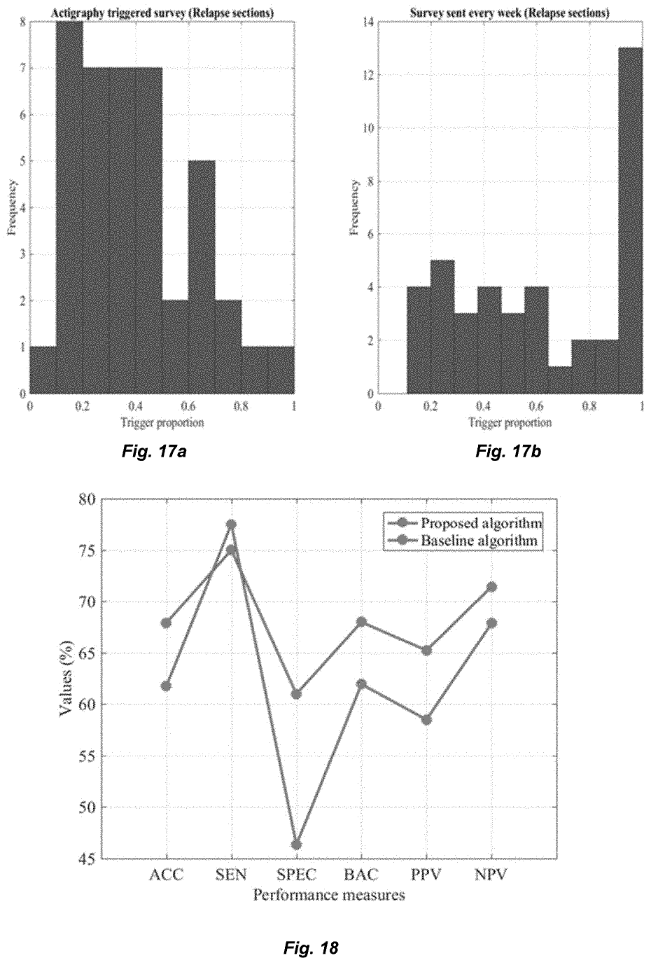

FIG. 15a shows data corresponding to a frequency of patients that are administered self-report tests over various trigger proportions, where the self-report surveys are administered to patients when determined by actigraphy data as described above in Example I. The data shown in FIG. 9a has a two-sample Kolmogorov-Smirnov goodness-of-fit hypothesis test P-value≤0.001 indicating the distributional difference in trigger proportion distribution. FIG. 9b shows data corresponding to frequency of patients that are administered self-report tests over various trigger proportions, where the self-report tests are administered on a weekly basis. As can be seen in FIG. 9a, a large frequency of patients that are administered self-report tests as directed by actigraphy showed a lower trigger proportion (e.g., 0.2) as compared to FIG. 9b where the largest frequency is for a trigger proportion of 1.0. The data shown in FIGS. 9a and 9b demonstrates that self-report tests are administered less when such tests are triggered by patient actigraphy data and thus, reducing burden to and promote compliance from patients. FIG. 10a shows a subset of the data shown in FIG. 9a for time frames where the patients are in remission and not approaching relapse. Similarly, FIG. 10b shows a subset of the data shown in FIG. 9b for time frames where the patients are in remission and not approaching relapse. FIG. 11a shows a subset of the data shown in FIG. 9a for time frames where the patients are approaching relapse. Similarly, FIG. 11b shows a subset of the data shown in FIG. 9b for time frames where the patients are approaching relapse. FIG. 12 shows performance metrics for the method of determining relapse of depression using actigraphy data in Example III. In FIG. 12, ACC indicates Accuracy, SEN indicates Sensitivity, SPEC indicates specificity, BAC indicates Balanced Accuracy, PPV indicates Positive Predictive Value, and NPV indicates Negative Predictive Value.

### Example IV

In Example IV, exemplary actigraphy devices 200 were provided to 41 patients who subsequently relapsed. Data for Example IV were collected in a similar manner as described above for Example I. The actigraphy devices 200 collected actigraphy data and the computing device 300 collected the patient's quantitative assessment of perception of sleep in response to the two self-report assessment questions described above in Example I. The data was analyzed by the processor 302 of the computing device 300 to detect and/or predict onset of relapse of depression in the patient. Example IV utilizes a similar method as described above in Example I, except as noted below, using an SVM anomaly detector and is iterated on a weekly basis. Example IV utilizes method 600 and administers the same two self-report tests as Example I via the computing device 300 and further determines whether the patient is likely to have experienced onset of relapse of depression by analyzing the current and prior results from the most recent three weeks to determine whether the patient is at risk of a relapse as show below in Table 5.

TABLE 5

| Most Recent 3- | |
|---|---|
| Weeks of Data | Determine as at Risk of Relapse When |
| 1 week of test | Current QIDS-SR16 score ≥11 or Current GAD-7 |
| data available | score ≥10 |
| 2 weeks of test | Current QIDS-SR16 score ≥11 or Current GAD-7 |
| data available | score >=10 or QIDS-SR16 score ≥9 (for 2 weeks) |
| | or GAD-7 score ≥5 (for 2 weeks) |
| 3 weeks of test | Current QIDS-SR16 ≥11 score or Current GAD-7 |
| data available | score ≥10 or QIDS-SR16 score ≥5 (for at least 2 |
| | weeks) or GAD-7 score ≥5 (for at least 2 weeks) |

It is noted that in Table 5, the number of weeks of test data available refers to the number of most recent consecutive weeks that QIDS-SR16 and/or GAD-7 scores are available within the most recent three-weeks of data. Performance metrics for Example IV is shown below in Table 6.

TABLE 6

| Performance Metrics | Values |
|---|---|
| True Positive | 29 |
| True Negative | 25 |
| False Positive | 16 |
| False Negative | 10 |
| Accuracy | 0.68 |
| Sensitivity | 0.74 |
| Specificity | 0.61 |
| Balanced Accuracy (BAC) | 0.68 |
| Positive Predictive Value (PPV) | 0.6 |
| Negative Predictive Value (NPV) | 0.71 |
| DOR | 4.53 |
| F₁-measure | 0.69 |

FIG. 8 shows data corresponding to proportion of the group of 30 true positive patients determined as likely to have relapsed over amount of time prior to actual onset of relapse as determined by the MADRS method described in Example I. The data of FIG. 8 shows that 83% of the 30 true positive patients were identified as likely to have relapsed using actigraphy data 7 days or more earlier than actual relapse onset.

### Example V

In Example V, the method 1000 of FIG. 8 using an Enc-Dec AD and identifying anomalies based on a window of 14-day (w=14 days) was evaluated using data collected from 211 MDD subjects. Data from the 211 MDD subjects include longitudinal assessment of self-reported symptoms (measured weekly, biweekly, etc.) and continuously collected actigraphy data for more than a year or until a first episode of relapse of depression. The subjects also adhered to regular bimonthly clinical visits. Each of the subjects completed self-reported assessments using a smartphone at and between clinical visits at regular frequencies (e.g., from weekly to bimonthly). Actigraphy data was continuously collected from each subject using a device worn on the nondominant wrist that measures acceleration in the direction parallel to the face of the device with a continuous sampling of 32 Hz, such as, for example Philips Actiwatch by Philips Respironics. The raw acceleration data recorded by the device were integrated into counts in 15-s epochs (actigraphy data) that reflect the movement amplitude. In order to minimize the epochs with zero activity counts, a minute resolution activity counts data stream was created by summing the 15-s epoch within each minute resulting in 1440 activity counts data points in a day.

Each clinical visit was labelled as relapse if the patient meets based on any one of the following criteria listed below:
(1) MADRS total score ≥22 at a study visit and a symptom worsening confirmed over an approximately 1 to 2-week interval.
(2) If a subject receives a MADRS rating ≥22 at a study visit (scheduled or unscheduled), an additional visit (i.e. the Relapse Verification visit) will be scheduled within 1 to 2 weeks
   subjects whose MADRS rating is ≥22 at the relapse verification visit will be considered to have relapsed
      CGI-S change from baseline ≥2 at the relapse verification visit or medication change happened during + or −14 days from the study visit will be considered to have relapsed.
(3) Hospitalization for worsening of depression
(4) Suicidal ideation with intent, or suicidal behavior
(5) Decision by mental health provider

If the clinical visit does not meet the above criteria, it was labelled as non-relapse. The labels were assumed to persist until the day after the previous clinical visit. Among the 211 MDD subjects, a total of 1190 visits (1140 non-relapse visits and 50 relapse visits) corresponding to 50 relapse subjects and 161 non-relapse subjects were evaluated. The first clinical visit after initiating collection of actigraphy data in all subjects is used to train the Enc-Dec AD for each subject and therefore not included in the 1190 visits for evaluation. In addition, the last visit evaluated for relapse subjects is their relapse visit. Therefore, the evaluations of Example V is until a first relapse and multiple relapses are not considered in this example.

Distribution of the 211 MDD subjects by each of the relapse criteria identified above is provided in Table 7.

TABLE 7

| Relapse criteria | N (%) |
|---|---|
| MADRS total score 2 22 at a study visit and a | 45 (90) |
| symptom worsening confirmed over an | |
| approximately 1 to 2-week interval. | |
| Hospitalization for worsening of depression | 2 (4) |
| Suicidal ideation with intent, or suicidal behavior | 1 (2) |
| Investigator’s decision | 2 (4) |

Characteristics of the 211 MDD subjects are summarized in Table 8 below.

TABLE 8

| | Relapse | | Non-relapse | |
|---|---|---|---|---|
| | subjects | | subjects | |
| Characteristic | (N = 50) (%) | | (N = 161) (%) | |
| Age (years), mean (SD) | 45.56 | (11.67) | 43 | (13.16) |

Age category (years), N (%)

| | | | | |
|---|---|---|---|---|
| Age 18-44 | 21 | (42) | 82 | (50.93) |
| Age ≥45 | 29 | (58) | 79 | (49.06) |
| Sex, N (%) | | | | |
| Men | 14 | (28) | 48 | (29.81) |
| Women | 36 | (72) | 113 | (70.19) |

Race, N (%)

| | | | | |
|---|---|---|---|---|
| White | 37 | (74) | 129 | (80.12) |
| Black or African American | 9 | (18) | 23 | (14.29) |
| Asian | 1 | (2) | 5 | (3.1) |
| Native Hawaiian or other | 0 | (0) | 1 | (0.62) |
| Pacific Islander | | | | |
| Unknown | 2 | (4) | 2 | (1.24) |
| Other | 1 | (2) | 1 | (0.62) |

Ethnicity, N (%)

| | | | | |
|---|---|---|---|---|
| Hispanic or Latino | 3 | (6) | 7 | (4.34) |
| Not Hispanic or Latino | 45 | (90) | 154 | (95.65) |
| Not reported | 2 | (4) | 0 | (0) |
| Weight (kg), mean (SD) | 83.96 | (25.48) | 31.0587.55 | (22.28) |
| BMI (kg/m²), mean (SD) | 29.44 | (8.27) | 31.0630.97 | (7.92) |

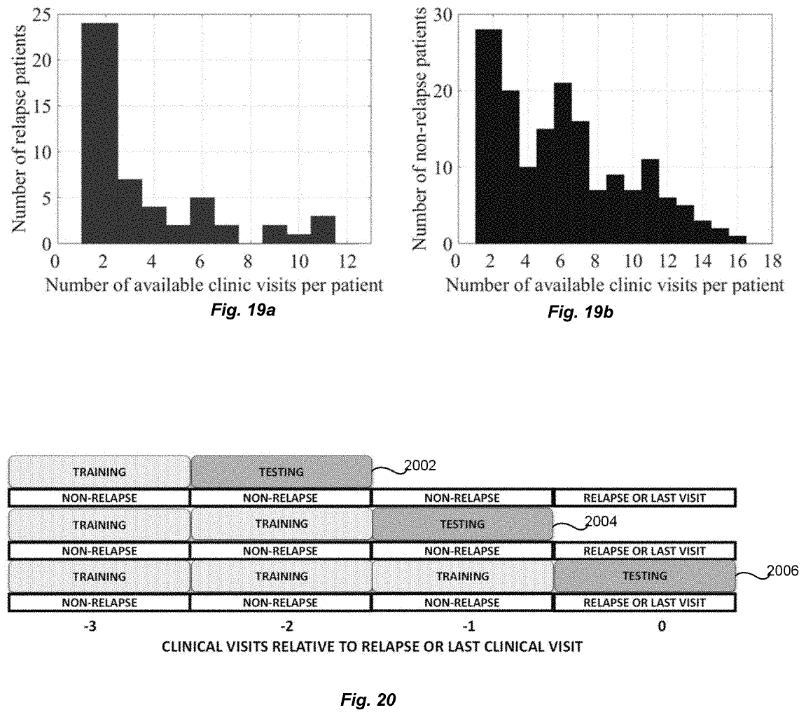

Additionally, FIGS. 19a and 19b show all the available visits for relapse and non-relapse subjects, respectively.

The actigraphy features were analyzed to extract features for DFA, MFDFA and Sample Entropy. These features are believed to be less affected by uncontrolled daily schedules and environmental conditions which can lead to an objective assessment of circadian ultradian rhythms and complexity in activity patterns. In Example V, the DFA feature was determined using a second order polynomial function to detrend the actigraphy data to eliminate the effect of possible linear trends in the data. To ensure reliable estimation of F(n) at a time scale n, the most recent continuous days of activity (at least 2 consecutive days) with no gaps >72 minute (5% of 1440 minutes of activity counts in a day) for each day were used. Fluctuation exponent (a) at two different time scales as α₁ during 10 (i.e., 10 data points with epoch length of 1 min) minutes to 90 minutes and α₂ during 120 minutes to 600 minutes respectively to capture the distinct regions of activity dynamics. The MFDFA features are extracted using values of q varied from −5 to 5 in increments of 0.1 and s is varied from 10 minutes to 600 minutes. Sample entropy features are extracted for each day and each of four epochs, morning (6 am to 12 pm), afternoon (12 pm to 6 pm), evening (6 pm to 12 am), and night (12 am to 6 am). More particularly, SaEn is determined as the median SaEn value (on z-score activity counts) across the last 7 days of activity counts (without any identified gaps).

All the actigraphy features were computed for each day with the last 7 days of continuous activity counts with a minimum requirement of at least 2 days of continuous activity counts. The activity counts were further subjected to signal quality checks to detect the following: (i) isolation of huge spikes with amplitude going 10 standard deviations away from the global mean levels and (ii) sequence of zeros with duration >60 minutes. The identified data points or segments as labelled as gap and handled appropriately in feature computation.

Features extracted from actigraphy data in the initial period before the first bimonthly clinical visit are used to train an Enc-Dec AD. The Enc-Dec AD of the Example V are specified according to the parameters listed in Table 9.

TABLE 9

| Parameters | Values |
|---|---|
| Hidden layer | 1 |
| Number of hidden units | 5 |
| Sequence length | 14 |
| Training iterations | 100 |
| Learning rate | 0.001 |
| Batch size | 1 |
| Optimizer | Adam |
| Input dimension | 7 |

If the first bimonthly clinical visit determined that the patient had not relapsed into depression, then subsequently collected actigraphy data were analyzed according to method 1000 using the trained Enc-Dec AD to identify any anomalies. This process continued with every non-relapse visit to the clinical visit and the Enc-Dec AD was retrained after every visit indicating that the patient had not relapsed into depression. The retrained Enc-Dec AD was used to identify anomalies in data collected for a subsequent visit. For any identified anomalies, self-report symptomology questionnaires, VQIDS-SR5 and GAD-7, collected at the same time as the identified anomalies were evaluated to further assess according to step 1008 as a further confirmatory step for determining whether the anomalies indicate that the patient is likely to experience onset of relapse of depression. In Example V, an anomaly is determined as indicating that the patient is likely to experience onset of relapse of depression after analyzing data from self-report symptomology questionnaires during a week where an anomaly is detected and a week following the anomalous week. If data from self-report symptomology questionnaires are not available during a week where an anomaly is detected or during a week following the anomalous week, the anomaly is not determined as indicating that onset of relapse of depression is likely.

FIG. 20 shows exemplary timelines for three different clinical visits illustrating that the Enc-Dec AD of Example V is continuously trained on every non-relapse visit's actigraphy data and anomalies are predicted in subsequent visit actigraphy data. The top timeline 2002 shows a patient having at least 1 non-relapse visit and that prior actigraphy data is used as training data for analyzing the testing data. As shown in the middle timeline 2004, the testing data from the previous visit (shown in timeline 2002) becomes training data for the visit illustrated in timeline 2002. Similarly in the bottom timeline 2006, the testing data from the previous visits (shown in timelines 2002 and 2004) become training data for the visit illustrated in timeline 2006. For each relapse and non-relapse subject, the process is continued until the first relapse, which is the last clinical visit for the relapsed patient in Example V.

Performance metrics for determining non-relapse vs relapse in Example V are shown below in Table 10.

TABLE 10

| | |
|---|---|
| # periods of relapse | 50 |
| # periods of non-relapse | 1140 |
| SEN (Sensitivity) | 0.66 |
| SPEC (Specificity) | 0.82 |
| BAC (Balanced Accuracy) | 0.74 |
| PPV (Positive Predictive Value) | 0.14 |
| NPV (Negative Predictive Value) | 0.98 |
| FAR (False Alarm Rate) | 0.18 |

As shown above in Table 10, Example V achieved sensitivity of 0.66, specificity of 0.82 and balanced accuracy of 0.74 in predicting relapse. The observed prevalence of relapse is 4.2% and Example V achieved a positive predictive value of 0.14 and negative predictive value of 0.98. Example V also achieved an overall false alarm rate (FAR) of 0.18 (FAR of 0.28 for relapse subjects and FAR of 0.16 for non-relapse subjects) among relapse and non-relapse subjects.

As shown above in Table 10, Example V has a false alarm rate of 0.18 across relapse and non-relapse subjects. The false alarm rate in relapse subjects is 0.28 and in non-relapse subjects is 0.16 indicating the ability of Example V to detect and/or predict relapse of depression more often in relapse subjects and thus, indicating that a determination by Example V as likely to experience a relapse of depression may eventually lead to a relapse event.

Performance metrics for determining non-relapse vs relapse in Example V, as shown in Table 10, are compared to performance metrics for detection of anomalies in the test actigraphy data alone or identification of relapse based on weekly collection of self-report test data alone.

TABLE 11

| | Anomaly | Self-report | Example |
|---|---|---|---|
| | detection | test | V |
| | only | data only | (Table 10) |
| SEN (Sensitivity) | 0.86 | 0.76 | 0.66 |
| SPEC (Specificity) | 0.14 | 0.69 | 0.82 |
| BAC (Balanced Accuracy) | 0.50 | 0.73 | 0.74 |
| PPV (Positive Predictive Value) | 0.04 | 0.10 | 0.14 |
| NPV (Negative Predictive Value) | 0.96 | 0.99 | 0.98 |
| FAR (False Alarm Rate) | 0.82 | 0.29 | 0.18 |
| TOD (Time to detection)(median) | 28 | 28 | 21 |

As can be seen in Table 11, Example V provides comparable sensitivity, while significantly increasing specificity as compared to anomaly detection alone or self-report test data alone. Notably, Example V provides a significant reduction in the FAR as compared to anomaly detection or weekly collection of self-report test data alone. Example V provides a reduction in FAR that could not be achieved by each of these components alone. As shown in Table 11, Example V, which utilizes a specific time aligned combination of anomaly detection and self-report test data, provides an unexpectedly greater (or synergistic) reduction in FAR than a combination of two separate analyses of anomaly detection and self-report test data. This data suggests that for any subjects who are at risk of relapse based on their baseline assessment, a positive prediction by the framework would most likely lead to an eventual relapse unless intervened earlier. The method of Example V was able to identify that the patient is likely to experience onset of relapse of depression at a median of 21 days before onset of depression, which provides a window of opportunity to adjust treatment for depression before onset of relapse of depression.

In view of the reduction in FAR, the method of Example V also significantly reduces provider burden as compared to anomaly detection alone or self-report test data alone. As can be seen in Table 12 below

TABLE 12

| | Patient burden (%)* | Provider burden (%)** |
|---|---|---|
| Anomaly detection only | 0 | 85.6 |
| Self-report test data only | 80.6 | 32.7 |
| Example V | 37.6 | 20.3 |

*Total weekly assessments = 8237;
**Total bimonthly visits = 1190

Table 12 shows provider burden represented by a percentage of the bimonthly preemptive visits scheduled that corresponded to a determination that the patient is likely to experience onset of relapse of depression based on anomaly detection alone, self-report test data alone, or the method of Example V. Notably, Example V provides a significant reduction in provider burden as compared to anomaly detection or weekly collection of self-report test data alone. Example V provides a reduction in provider burden that could not be achieved by each of these components alone. As shown in Table 12, Example V, which utilizes a specific time aligned combination of anomaly detection and self-report test data, provides an unexpectedly greater (or synergistic) reduction in provider burden than a combination of two separate analyses of anomaly detection and self-report test data.

Table 12 also shows patient burden represented by a percentage of total scheduled weekly self-report assessments that correspond to a determination that self-report data would be analyzed (i.e., those that are used in the analysis) in determining whether the patient is likely to experience onset of relapse. For weekly self-report assessments scheduled after a determination that the patient is likely to experience onset of relapse, those assessments not part of the percentage representing patient burden. This data is collected across the study population of 211 subjects followed for year or more. As shown in Table 12, selectively administering self-report tests according to Example V also significantly reduces the patient burden and thereby providing an effective way to monitor patients that is sufficiently sensitive, with low FAR, and with low burdens on both the patient and provider.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. A computer-implemented method for detecting or predicting return of depression in a patient comprising:
(i) obtaining, from a wearable device worn by the patient, training data of the patient over a training period, wherein the training data comprises training actigraphy data corresponding to movement of the patient over the training period, and the training period is during a time period when the patient has not experienced onset of return of depression;
(ii) training an anomaly detector using the training data, wherein the anomaly detector is configured to identify deviations from the training data;
(iii) obtaining, from the wearable device, test data of the patient during a test period after the training period, the test data comprising test actigraphy data corresponding to movement of the patient after the training period;
(iv) extracting a plurality of features from the test data to generate test feature data, wherein the features correspond to metrics for at least one of activity, sleep, circadian rhythm, and multifractal dynamics;
(v) analyzing the test feature data using the anomaly detector to compare the test feature data to the training data;
(vi) administering a self-report test to the patient to obtain a plurality of inputs from the patient when the anomaly detector determines that the test feature data is likely an anomaly compared to the training actigraphy data; and
(vii) analyzing the plurality of inputs from the patient to determine whether the patient is likely to experience onset of return of depression.

2. The method of claim 1, further comprising:
(viii) updating the training data to include the test data, and repeating steps (ii) to (vii) until the patient is determined to have returned into depression.

3. The method of claim 2, wherein steps (ii) to (vii) are repeated weekly.

4. The method of claim 1, wherein the training data further comprises data corresponding to self-report characteristics of physical behavior of the patient over the training period, and the test data further comprises data corresponding to self-report characteristics of physical behavior of the patient during the test period.

5. The method of claim 1, wherein step (vi) comprises
displaying, via a user interface, a plurality of self-report survey questions to the patient, and
receiving, via the user interface, the plurality of inputs from the patient in response to the self-report survey questions.

6. The method of claim 1, wherein step (vii) comprises:
analyzing the plurality of inputs to generate a resulting score for the self-report test, and
comparing the resulting score to at least one threshold value to determine whether the patient is likely to experience onset of return of depression.

7. The method of claim 1, wherein the anomaly detector is a one-class support vector machine module.

8. The method of claim 1, wherein the anomaly detector is an isolation forest module.

9. The method of claim 1, wherein the training period is at least 3 months.

10. The method of claim 5, wherein the plurality of self-report survey questions corresponds to symptoms of depression, and the plurality of inputs from the patient corresponds to a rating on a numerical scale for each symptom.

11. The method of claim 1, further comprising:
adjusting a dosage of an antidepressant administered to the patient when the patient is determined as likely to experience onset of return of depression.

12. The method of claim 1, further comprising:
increasing a dosage of an antidepressant administered to the patient when the patient is determined as likely to experience onset of return of depression.

13. A system for detecting or predicting return of depression in a patient comprising:
a wearable device comprising at least one accelerometer configured to detect movement of the patient, the wearable device configured to generate actigraphy data corresponding to movement of the patient; and
a computing device operably connected to the wearable device to receive actigraphy data from the wearable device, the computing device comprising:
a user interface for displaying output and receiving input from the patient, and
a processor and a non-transitory computer readable storage medium including a set of instructions executable by the processor, the set of instructions operable to:
obtain, from the wearable device, training actigraphy data corresponding to movement of the patient over a training period, wherein the training period is during a time period when the patient has not experienced onset of return of depression,
train an anomaly detector using training data comprising the training actigraphy data, wherein the anomaly detector is configured to identify deviations from the training data,
obtaining, from the wearable device, test actigraphy data corresponding to movement of the patient after the training period,
extract a plurality of features from the test actigraphy data to generate test feature data, wherein the features correspond to metrics for at least one of activity, sleep, circadian rhythm, and multifractal dynamics,
analyze the test feature data using the anomaly detector to compare the test feature data to the training data,
direct the user interface to display a plurality of self-report survey questions to the patient,
receive, via the user interface, the plurality of inputs from the patient in response to the self-report survey questions, and
analyze the plurality of inputs from the patient to determine whether the patient is likely to experience onset of return of depression.

14. The system of claim 13, wherein the actigraphy device, in an operating configuration, is configured for wearing around a wrist of the patient.

15. The system of claim 13, wherein the user interface is a touch screen.

16. The system of claim 13, wherein the computing device is selected from a group consisting of a mobile computing device, a smart phone, and a computing tablet.

17. The system of claim 13, wherein the anomaly detector is a one-class support vector machine module.

18. The system of claim 13, wherein the anomaly detector is an isolation forest module.

19. The system of claim 13, wherein the plurality of self-report survey questions corresponds to symptoms of depression, and the plurality of inputs from the patient corresponds to rating on a numerical scale of each correspond symptom.

20. The system of claim 13, wherein the set of instructions further comprises instructions operable to direct an output indicating an adjustment for a dosage of an antidepressant administered to the patient when the patient is determined by the computing device as likely to experience onset of return of depression.

21. A computer-implemented method for detecting or predicting return of depression in a patient comprising:
(i) obtaining, from a wearable device worn by the patient, training data of the patient over a training period, wherein the training data comprises training actigraphy data corresponding to movement of the patient over the training period, and the training period is during a time period when the patient has not experienced onset of return of depression;
(ii) training an anomaly detector using the training data, wherein the anomaly detector is configured to identify deviations from the training data;
(iii) obtaining, from the wearable device, test data of the patient during a test period, at least a portion of the test period being after the training period, the test data comprising test actigraphy data corresponding to movement of the patient after the training period;
(iv) extracting a plurality of features from the test data to generate test feature data, wherein the features correspond to metrics for at least one of monofractal patterns, multifractal dynamics and sample entropy;
(v) analyzing the test feature data using the anomaly detector to compare the test feature data to the training data to detect an anomaly in the test feature data; and
(vi) analyzing self-report test data to determine whether the patient is likely to experience onset of return of depression when an anomaly is detected in the test feature data, wherein the self-report test data is generated from a plurality of inputs from the patient in response to a self-report test.

22. The method of claim 21, wherein the self-reported test is collected from a time concurrent with the detected anomaly.

23. The method of claim 21 or 22, wherein the self-reported test is collected from the patient after an anomaly is detected.

24. The method of claim 21, further comprising:
(vii) updating the training data to include the test data, and repeating steps (ii) to (vi) until the patient is determined to have returned into depression.

25. The method of claim 24, wherein steps (ii) to (vii) are repeated continuously until the patient is determined to have returned into depression.

26. The method of claim 21, wherein step (vi) comprises:
analyzing the self-report test data to generate a resulting score for the self-report test, and
comparing the resulting score to at least one threshold value to determine whether the patient is likely to experience onset of return of depression.

27. The method of claim 21, wherein the anomaly detector utilizes a long short-term memory (LSTM) neural network, the anomaly detector comprising an encoder and a decoder.

28. The method of claim 27, wherein step (v) comprises:
identifying non-anomalous sections of the test feature data using a first anomaly threshold;
determining potential anomalous instances in the test feature data using a second anomaly threshold, wherein the second anomaly threshold is determined based on the non-anomalous sections;
pruning the potential anomalous instances based on a percent decrease for each potential anomalous instance to identify the anomaly in the test feature data.

29. The method of claim 21, wherein the training period is at least 14 days.

30. The method of claim 21, further comprising:
adjusting a dosage of an antidepressant administered to the patient when the patient is determined as likely to experience onset of return of depression.

31. The method of claim 21, further comprising:
increasing a dosage of an antidepressant administered to the patient when the patient is determined as likely to experience onset of return of depression.

32. A system for detecting or predicting return of depression in a patient comprising:
a wearable device comprising at least one accelerometer configured to detect movement of the patient, the wearable device configured to generate actigraphy data corresponding to movement of the patient; and
a computing device operably connected to the wearable actigraphy device to receive actigraphy data from the wearable device, the computing device comprising:
a user interface for displaying output and receiving input from the patient, and
a processor and a non-transitory computer readable storage medium including a set of instructions executable by the processor, the set of instructions operable to:
obtain, from the wearable device, training actigraphy data corresponding to movement of the patient over a training period, wherein the training period is during a time period when the patient has not experienced onset of return of depression,
train an anomaly detector using training data comprising the training actigraphy data, wherein the anomaly detector is configured to identify deviations from the training data,
obtaining, from the wearable device, test actigraphy data corresponding to movement of the patient during a test period, at least a portion of the test period being after the training period,
extract a plurality of features from the test actigraphy data to generate test feature data, wherein the features correspond to metrics for at least one of activity, for at least one of monofractal patterns, multifractal dynamics and sample entropy,
analyze the test feature data using the anomaly detector to compare the test feature data to the training data to detect an anomaly in the test feature data,
analyze self-report test data to determine whether the patient is likely to experience onset of return of depression when an anomaly is detected in the test feature data,
wherein the self-report test data is generated from a plurality of inputs received from the patient by the user interface in response to a self-report test comprising a plurality of self-report survey questions displayed on the user interface.

33. The system of claim 32, wherein the actigraphy device, in an operating configuration, is configured for wearing around a wrist of the patient.

34. The system of claim 32, wherein the user interface is a touch screen.

35. The system of claim 32, wherein the computing device is selected from a group consisting of a mobile computing device, a smart phone, and a computing tablet.

36. The system of claim 32, wherein the anomaly detector utilizes a long short-term memory (LSTM) neural network, the anomaly detector comprising an encoder and a decoder.

37. The system of claim 32, wherein the plurality of self-report survey questions corresponds to symptoms of depression, and the plurality of inputs from the patient corresponds to rating on a numerical scale of each correspond symptom.
